SEQUENCE LISTING (245 sequences)

SEQ ID NO: 1 - protein, 10 aa, Artificial Sequence
  Description of Artificial Sequence Synthetic peptide
XXXXXXXXXX

SEQ ID NO: 2 - protein, 10 aa, Artificial Sequence
  Description of Artificial Sequence Synthetic peptide
CXXXXXTXXC

SEQ ID NO: 3 - protein, 12 aa, Artificial Sequence
  Description of Artificial Sequence Synthetic peptide
YXCXXXXXTXXC

SEQ ID NO: 4 - protein, 16 aa, Artificial Sequence
  Description of Artificial Sequence Synthetic peptide
XYXCXXXXXTXXCXXX

SEQ ID NO: 5 - protein, 20 aa, Artificial Sequence
  Description of Artificial Sequence Synthetic peptide
GGTYACHFGKLTXVCKKQGG

SEQ ID NO: 6 - protein, 20 aa, Artificial Sequence
  Description of Artificial Sequence Synthetic peptide
GGTYSCHMGKLTXVCKKQGG

SEQ ID NO: 7 - protein, 20 aa, Artificial Sequence
  Description of Artificial Sequence Synthetic peptide
GGLYACHFGKLTXVCRKQGG

SEQ ID NO: 8 - protein, 20 aa, Artificial Sequence
  Description of Artificial Sequence Synthetic peptide
GGTYTCHFGKLTXVCKKLGG

SEQ ID NO: 9 - protein, 20 aa, Artificial Sequence
  Description of Artificial Sequence Synthetic peptide
GGLYACHFGKLTXICKKQGG

SEQ ID NO: 10 - protein, 20 aa, Artificial Sequence
  Description of Artificial Sequence Synthetic peptide
GGLYSCHFGKITXECKKQGG

SEQ ID NO: 11 - protein, 20 aa, Artificial Sequence
  Description of Artificial Sequence Synthetic peptide
GGTYACHFGKLTWVCKKQGG

SEQ ID NO: 12 - protein, 20 aa, Artificial Sequence
  Description of Artificial Sequence Synthetic peptide
GGTYSCHFGKLTWVCKKNGG

SEQ ID NO: 13 - protein, 20 aa, Artificial Sequence
  Description of Artificial Sequence Synthetic peptide
GGLYACHFGKLTXVCKKQGG

SEQ ID NO: 14 - protein, 20 aa, Artificial Sequence
  Description of Artificial Sequence Synthetic peptide
GGLYSCHMGKLTWVCKKQRG

SEQ ID NO: 15 - protein, 20 aa, Artificial Sequence
  Description of Artificial Sequence Synthetic peptide
GGTYLCHFGKLTWVCKKQGG

SEQ ID NO: 16 - protein, 20 aa, Artificial Sequence
  Description of Artificial Sequence Synthetic peptide
GGTLSCEFGKLTEVCDKQGG

SEQ ID NO: 17 - protein, 20 aa, Artificial Sequence
  Description of Artificial Sequence Synthetic peptide
GGLYSCHFGKLTWVCKKQGG

SEQ ID NO: 18 - protein, 20 aa, Artificial Sequence
  Description of Artificial Sequence Synthetic peptide
GGTYHCHFGKLTWVCKKQGG

SEQ ID NO: 19 - protein, 20 aa, Artificial Sequence
  Description of Artificial Sequence Synthetic peptide
GGLYSCHFGKITXVCKKQGG

SEQ ID NO: 20 - protein, 20 aa, Artificial Sequence
  Description of Artificial Sequence Synthetic peptide
GGLYSCHMGKITWVCRKQGG

SEQ ID NO: 21 - protein, 20 aa, Artificial Sequence
  Description of Artificial Sequence Synthetic peptide
GGTYSCHFGKITXVCKKQGG

SEQ ID NO: 22 - protein, 20 aa, Artificial Sequence
  Description of Artificial Sequence Synthetic peptide
GGTYSCHFGKLTXVCQKQGG

SEQ ID NO: 23 - protein, 20 aa, Artificial Sequence
  Description of Artificial Sequence Synthetic peptide
GGLYSCHFGKITWVCKKQRG

SEQ ID NO: 24 - protein, 20 aa, Artificial Sequence
  Description of Artificial Sequence Synthetic peptide
GGLYSCHMGKLTWVCRKQGG

SEQ ID NO: 25 - protein, 20 aa, Artificial Sequence
  Description of Artificial Sequence Synthetic peptide
GGLYACHFGKITWVCRKQGG

SEQ ID NO: 26 - protein, 20 aa, Artificial Sequence
  Description of Artificial Sequence Synthetic peptide
GGLYACHFGKLTWVCRKQRG

SEQ ID NO: 27 - protein, 20 aa, Artificial Sequence
  Description of Artificial Sequence Synthetic peptide
GGLYSCHMGKLTXDCKKQGG

SEQ ID NO: 28 - protein, 20 aa, Artificial Sequence
  Description of Artificial Sequence Synthetic peptide
GGLYACHFGKLTWVCRKLGG

SEQ ID NO: 29 - protein, 20 aa, Artificial Sequence
  Description of Artificial Sequence Synthetic peptide
GGLYSCHFGKLTXVCKKLGG

SEQ ID NO: 30 - protein, 20 aa, Artificial Sequence
  Description of Artificial Sequence Synthetic peptide
GGTYSCHFGKLTWVCSKQGG

SEQ ID NO: 31 - protein, 20 aa, Artificial Sequence
  Description of Artificial Sequence Synthetic peptide
GGLYSCHFGKITWVCKKQGG

SEQ ID NO: 32 - protein, 20 aa, Artificial Sequence
  Description of Artificial Sequence Synthetic peptide
GGDYSCHFGKLTWVCKKQGG

SEQ ID NO: 33 - protein, 20 aa, Artificial Sequence
  Description of Artificial Sequence Synthetic peptide
GGLYSCHFGKLTXVCQKQGG

SEQ ID NO: 34 - protein, 20 aa, Artificial Sequence
  Description of Artificial Sequence Synthetic peptide
GGTYSCHFGKLTWVCQKQRG

SEQ ID NO: 35 - protein, 20 aa, Artificial Sequence
  Description of Artificial Sequence Synthetic peptide
GGTYSCHFGKLTWVCGKQGG

SEQ ID NO: 36 - protein, 20 aa, Artificial Sequence
  Description of Artificial Sequence Synthetic peptide
GGLYSCHFGKLTXVCQKQGG

SEQ ID NO: 37 - protein, 20 aa, Artificial Sequence
  Description of Artificial Sequence Synthetic peptide
GGTYTCHMGKLTWVCKKQGG

SEQ ID NO: 38 - protein, 20 aa, Artificial Sequence
  Description of Artificial Sequence Synthetic peptide
GGTYSCHFGKLTWVCKKQGG

SEQ ID NO: 39 - protein, 20 aa, Artificial Sequence
  Description of Artificial Sequence Synthetic peptide
GGLYSCHFGKLTWVCRKQRG

SEQ ID NO: 40 - protein, 20 aa, Artificial Sequence
  Description of Artificial Sequence Synthetic peptide
GGTYSCHFGKLTXVCKKQRG

SEQ ID NO: 41 - protein, 20 aa, Artificial Sequence
  Description of Artificial Sequence Synthetic peptide
GGLYACHFGKLTWVCQKQGG

SEQ ID NO: 42 - protein, 20 aa, Artificial Sequence
  Description of Artificial Sequence Synthetic peptide
GGLYACHFGKLTWDCQKQGG

SEQ ID NO: 43 - protein, 20 aa, Artificial Sequence
  Description of Artificial Sequence Synthetic peptide
GGTYSCHFGKLTWVCKKAGG

SEQ ID NO: 44 - protein, 20 aa, Artificial Sequence
  Description of Artificial Sequence Synthetic peptide
GGTYSCHFGKLTWICKKQGG

SEQ ID NO: 45 - protein, 20 aa, Artificial Sequence
  Description of Artificial Sequence Synthetic peptide
GGTYSCHFGKLTWVCLKQGG

SEQ ID NO: 46 - protein, 20 aa, Artificial Sequence
  Description of Artificial Sequence Synthetic peptide
GGVYSCHFGKLTWVCKKQGG

SEQ ID NO: 47 - protein, 20 aa, Artificial Sequence
  Description of Artificial Sequence Synthetic peptide
GGTYTCHFGKLTXVCKKQGG

SEQ ID NO: 48 - protein, 20 aa, Artificial Sequence
  Description of Artificial Sequence Synthetic peptide
GGTYSCHFGKITWVCKKQRG

SEQ ID NO: 49 - protein, 20 aa, Artificial Sequence
  Description of Artificial Sequence Synthetic peptide
GGLYSCHFGKLTWVCRKLGG

SEQ ID NO: 50 - protein, 20 aa, Artificial Sequence
  Description of Artificial Sequence Synthetic peptide
GGTLSCDFGGLTWVCEKQEG

SEQ ID NO: 51 - protein, 20 aa, Artificial Sequence
  Description of Artificial Sequence Synthetic peptide
GGTYSCHFGKLTXVCKKLGG

SEQ ID NO: 52 - protein, 20 aa, Artificial Sequence
  Description of Artificial Sequence Synthetic peptide
GGLYSCHMGKLTWVCKKLGG

SEQ ID NO: 53 - protein, 20 aa, Artificial Sequence
  Description of Artificial Sequence Synthetic peptide
GGLYSCHMGKLTWDCKKQRG

SEQ ID NO: 54 - protein, 20 aa, Artificial Sequence
  Description of Artificial Sequence Synthetic peptide
GGTYSCHFGKLTWLCKKQGG

SEQ ID NO: 55 - protein, 20 aa, Artificial Sequence
  Description of Artificial Sequence Synthetic peptide
GGLYSCHFGKLTXVCRKQRG

SEQ ID NO: 56 - protein, 20 aa, Artificial Sequence
  Description of Artificial Sequence Synthetic peptide
GGLYACHMGKLTWDCKKQGG

SEQ ID NO: 57 - protein, 20 aa, Artificial Sequence
  Description of Artificial Sequence Synthetic peptide
GGTYTCHFGKLTXVCKKLGG

SEQ ID NO: 58 - protein, 20 aa, Artificial Sequence
  Description of Artificial Sequence Synthetic peptide
GGTYACHMGKLTWVCKKQGG

SEQ ID NO: 59 - protein, 20 aa, Artificial Sequence
  Description of Artificial Sequence Synthetic peptide
GGTGSCEFGGLAKVCEKQEG

SEQ ID NO: 60 - protein, 20 aa, Artificial Sequence
  Description of Artificial Sequence Synthetic peptide
GGMYSCHFGKLTWVCKKQGG

SEQ ID NO: 61 - protein, 20 aa, Artificial Sequence
  Description of Artificial Sequence Synthetic peptide
GGLYSCHMGKLTWVCKKQGG

SEQ ID NO: 62 - protein, 20 aa, Artificial Sequence
  Description of Artificial Sequence Synthetic peptide
GGLYSCHFGKITWVCQKQGG

SEQ ID NO: 63 - protein, 20 aa, Artificial Sequence
  Description of Artificial Sequence Synthetic peptide
GGTYSCHMGKLTXVCKKQGG

SEQ ID NO: 64 - protein, 20 aa, Artificial Sequence
  Description of Artificial Sequence Synthetic peptide
GGTYSCHFGKITWVCKKLGG

SEQ ID NO: 65 - protein, 20 aa, Artificial Sequence
  Description of Artificial Sequence Synthetic peptide
GGTLSCDFGKLTWVCEKQGG

SEQ ID NO: 66 - protein, 20 aa, Artificial Sequence
  Description of Artificial Sequence Synthetic peptide
GGTYSCHFGKITXVCKKQGG

SEQ ID NO: 67 - protein, 20 aa, Artificial Sequence
  Description of Artificial Sequence Synthetic peptide
GGTYSCHMGKITWVCKKQGG

SEQ ID NO: 68 - protein, 20 aa, Artificial Sequence
  Description of Artificial Sequence Synthetic peptide
GGTYSCDPGGLFWVCEKQGG

SEQ ID NO: 69 - protein, 20 aa, Artificial Sequence
  Description of Artificial Sequence Synthetic peptide
GGLYACHMGKITXVCQKLRG

SEQ ID NO: 70 - protein, 20 aa, Artificial Sequence
  Description of Artificial Sequence Synthetic peptide
GGTYTCHFGKLTWVCKKLGG

SEQ ID NO: 71 - protein, 20 aa, Artificial Sequence
  Description of Artificial Sequence Synthetic peptide
GGTYSCHFGKLTXVCKKQRG

SEQ ID NO: 72 - protein, 20 aa, Artificial Sequence
  Description of Artificial Sequence Synthetic peptide
GGTYSCHMGKLTWVCKKQGG

SEQ ID NO: 73 - protein, 20 aa, Artificial Sequence
  Description of Artificial Sequence Synthetic peptide
GGTYTCHMGKITWVCKKQGG

SEQ ID NO: 74 - protein, 20 aa, Artificial Sequence
  Description of Artificial Sequence Synthetic peptide
GGLYSCHFGKLTXVCKKLGG

SEQ ID NO: 75 - protein, 20 aa, Artificial Sequence
  Description of Artificial Sequence Synthetic peptide
GGTYACHFGKLTWVCKKLGG

SEQ ID NO: 76 - protein, 20 aa, Artificial Sequence
  Description of Artificial Sequence Synthetic peptide
GGLYACHFGKLTXVCKKQGG

SEQ ID NO: 77 - protein, 20 aa, Artificial Sequence
  Description of Artificial Sequence Synthetic peptide
GGTGSCEAGKLTDVCEKQDG

SEQ ID NO: 78 - protein, 20 aa, Artificial Sequence
  Description of Artificial Sequence Synthetic peptide
GGLYSCHFGKLTXVCRKQGG

SEQ ID NO: 79 - protein, 20 aa, Artificial Sequence
  Description of Artificial Sequence Synthetic peptide
GGTYTCHFGKLTWVCKKQGG

SEQ ID NO: 80 - protein, 20 aa, Artificial Sequence
  Description of Artificial Sequence Synthetic peptide
GGTLSCDFGKLGWVCKKQGG

SEQ ID NO: 81 - protein, 20 aa, Artificial Sequence
  Description of Artificial Sequence Synthetic peptide
GGTYSCHFGKLTWVCHKQGG

SEQ ID NO: 82 - protein, 20 aa, Artificial Sequence
  Description of Artificial Sequence Synthetic peptide
GGLYSCHMGKLTWVCQKQGG

SEQ ID NO: 83 - protein, 20 aa, Artificial Sequence
  Description of Artificial Sequence Synthetic peptide
GGTGSCEFGKLTEVCKKQGG

SEQ ID NO: 84 - protein, 20 aa, Artificial Sequence
  Description of Artificial Sequence Synthetic peptide
GGLYSCHFGKITWVCKKQAG

SEQ ID NO: 85 - protein, 20 aa, Artificial Sequence
  Description of Artificial Sequence Synthetic peptide
GGTYSCHFGKLTXVCKKLGG

SEQ ID NO: 86 - protein, 20 aa, Artificial Sequence
  Description of Artificial Sequence Synthetic peptide
GGLYACHMGKLTWVCRKQGG

SEQ ID NO: 87 - protein, 20 aa, Artificial Sequence
  Description of Artificial Sequence Synthetic peptide
GGTYSCHFGKLTWDCKKQGG

SEQ ID NO: 88 - protein, 20 aa, Artificial Sequence
  Description of Artificial Sequence Synthetic peptide
GGLYSCHMGKLTWLCKKLGG

SEQ ID NO: 89 - protein, 20 aa, Artificial Sequence
  Description of Artificial Sequence Synthetic peptide
GGTYTCHFGKLTWVCKKQRG

SEQ ID NO: 90 - protein, 20 aa, Artificial Sequence
  Description of Artificial Sequence Synthetic peptide
GGLYACHFGKLTWECKKLGG

SEQ ID NO: 91 - protein, 20 aa, Artificial Sequence
  Description of Artificial Sequence Synthetic peptide
GGTGSCDFGKLAWVCDKQEG

SEQ ID NO: 92 - protein, 20 aa, Artificial Sequence
  Description of Artificial Sequence Synthetic peptide
GGLYACHXGKLTWVCRKQGG

SEQ ID NO: 93 - protein, 20 aa, Artificial Sequence
  Description of Artificial Sequence Synthetic peptide
GGLYACHFGKLTWVCKKQRG

SEQ ID NO: 94 - protein, 20 aa, Artificial Sequence
  Description of Artificial Sequence Synthetic peptide
GGTGSCDAGKLTDVCDKQDG

SEQ ID NO: 95 - protein, 20 aa, Artificial Sequence
  Description of Artificial Sequence Synthetic peptide
GGLYSCHMGKITWLCKKQGG

SEQ ID NO: 96 - protein, 20 aa, Artificial Sequence
  Description of Artificial Sequence Synthetic peptide
GGLYACHXGKLTWVCKKQGG

SEQ ID NO: 97 - protein, 20 aa, Artificial Sequence
  Description of Artificial Sequence Synthetic peptide
GGTYSCHFGKITWVCKKQGG

SEQ ID NO: 98 - protein, 20 aa, Artificial Sequence
  Description of Artificial Sequence Synthetic peptide
GGLYSCHMGKLTWVCRKLGG

SEQ ID NO: 99 - protein, 20 aa, Artificial Sequence
  Description of Artificial Sequence Synthetic peptide
GGLYSCHFGKITWVCRKQAG

SEQ ID NO: 100 - protein, 20 aa, Artificial Sequence
  Description of Artificial Sequence Synthetic peptide
GGTGSCDFGKLTDVCAKQEG

SEQ ID NO: 101 - protein, 20 aa, Artificial Sequence
  Description of Artificial Sequence Synthetic peptide
GGTYSCRFGKLTWVCKKQGG

SEQ ID NO: 102 - protein, 20 aa, Artificial Sequence
  Description of Artificial Sequence Synthetic peptide
GGLYSCHFGKITWVCRKQGG

SEQ ID NO: 103 - protein, 20 aa, Artificial Sequence
  Description of Artificial Sequence Synthetic peptide
GGTYSCEGGKLGKVCEKQEG

SEQ ID NO: 104 - protein, 20 aa, Artificial Sequence
  Description of Artificial Sequence Synthetic peptide
GGTLSCDAGGLTKVCDKQDG

SEQ ID NO: 105 - protein, 20 aa, Artificial Sequence
  Description of Artificial Sequence Synthetic peptide
GGTLSCHPGKLTKVCKKQDG

SEQ ID NO: 106 - protein, 20 aa, Artificial Sequence
  Description of Artificial Sequence Synthetic peptide
GGTYTCHFGKITXVCKKQGG

SEQ ID NO: 107 - protein, 20 aa, Artificial Sequence
  Description of Artificial Sequence Synthetic peptide
GGTYACHFGKITWVCKKQGG

SEQ ID NO: 108 - protein, 20 aa, Artificial Sequence
  Description of Artificial Sequence Synthetic peptide
GGLYSCHFGKLTXVCKKQGG

SEQ ID NO: 109 - protein, 20 aa, Artificial Sequence
  Description of Artificial Sequence Synthetic peptide
GGLYSCHMGKLTWICQKQGG

SEQ ID NO: 110 - protein, 20 aa, Artificial Sequence
  Description of Artificial Sequence Synthetic peptide
GGTYSCHFGKLTWVCQKQGG

SEQ ID NO: 111 - protein, 20 aa, Artificial Sequence
  Description of Artificial Sequence Synthetic peptide
GGLYACHFGKLTXLCKKQGG

SEQ ID NO: 112 - protein, 20 aa, Artificial Sequence
  Description of Artificial Sequence Synthetic peptide
GGTLSCEFGKLFKVCEKQGG

SEQ ID NO: 113 - protein, 20 aa, Artificial Sequence
  Description of Artificial Sequence Synthetic peptide
GGTYTCHFGKITWVCKKQAG

SEQ ID NO: 114 - protein, 20 aa, Artificial Sequence
  Description of Artificial Sequence Synthetic peptide
GGLYSCHMGKLTWVCRKQRG

SEQ ID NO: 115 - protein, 20 aa, Artificial Sequence
  Description of Artificial Sequence Synthetic peptide
GGTYTCHFGKITWVCQKQGG

SEQ ID NO: 116 - protein, 20 aa, Artificial Sequence
  Description of Artificial Sequence Synthetic peptide
GGTYTCHMGKLTWVCKKLGG

SEQ ID NO: 117 - protein, 20 aa, Artificial Sequence
  Description of Artificial Sequence Synthetic peptide
GGTYTCHFGKITWVCKKQGG

SEQ ID NO: 118 - protein, 20 aa, Artificial Sequence
  Description of Artificial Sequence Synthetic peptide
GGTYSCHFGKLTWVCAKQGG

SEQ ID NO: 119 - protein, 20 aa, Artificial Sequence
  Description of Artificial Sequence Synthetic peptide
GGLYSCHFGKLTWVCQKQGG

SEQ ID NO: 120 - protein, 20 aa, Artificial Sequence
  Description of Artificial Sequence Synthetic peptide
GGLYSCHFGKLTWVCKKQRG

SEQ ID NO: 121 - protein, 20 aa, Artificial Sequence
  Description of Artificial Sequence Synthetic peptide
GGLYSCHFGKITXICKKQGG

SEQ ID NO: 122 - protein, 20 aa, Artificial Sequence
  Description of Artificial Sequence Synthetic peptide
GGTYTCHFGKLTXVCQKQGG

SEQ ID NO: 123 - protein, 20 aa, Artificial Sequence
  Description of Artificial Sequence Synthetic peptide
GGTGSCEPGKLTDVCEKQGG

SEQ ID NO: 124 - protein, 20 aa, Artificial Sequence
  Description of Artificial Sequence Synthetic peptide
GGTYTCHFGKITWVCKKQRG

SEQ ID NO: 125 - protein, 20 aa, Artificial Sequence
  Description of Artificial Sequence Synthetic peptide
GGTYTCHFGKITXVCKKQGG

SEQ ID NO: 126 - protein, 20 aa, Artificial Sequence
  Description of Artificial Sequence Synthetic peptide
GGTYTCHMGKLTWVCKKQRG

SEQ ID NO: 127 - protein, 20 aa, Artificial Sequence
  Description of Artificial Sequence Synthetic peptide
GGLYSCHMGKITWVCKKQGG

SEQ ID NO: 128 - protein, 20 aa, Artificial Sequence
  Description of Artificial Sequence Synthetic peptide
GGLYACHFGKITWECKKQGG

SEQ ID NO: 129 - protein, 20 aa, Artificial Sequence
  Description of Artificial Sequence Synthetic peptide
GGLYACHMGKITXVCQKLGG

SEQ ID NO: 130 - protein, 20 aa, Artificial Sequence
  Description of Artificial Sequence Synthetic peptide
GGLYSCHFGKLTXVCKKQRG

SEQ ID NO: 131 - protein, 20 aa, Artificial Sequence
  Description of Artificial Sequence Synthetic peptide
GGTYSCHFGKLTXVCKKQRG

SEQ ID NO: 132 - protein, 20 aa, Artificial Sequence
  Description of Artificial Sequence Synthetic peptide
GGLYACHFGKLTWVCRKQGG

SEQ ID NO: 133 - protein, 20 aa, Artificial Sequence
  Description of Artificial Sequence Synthetic peptide
GGTYSCEAGKLTKVCEKQEG

SEQ ID NO: 134 - protein, 20 aa, Artificial Sequence
  Description of Artificial Sequence Synthetic peptide
GGLYACHFGKLTWVCKKLGG

SEQ ID NO: 135 - protein, 20 aa, Artificial Sequence
  Description of Artificial Sequence Synthetic peptide
GGTYTCHFGKLTXVCKKQGG

SEQ ID NO: 136 - protein, 20 aa, Artificial Sequence
  Description of Artificial Sequence Synthetic peptide
GGLYSCHFGKLTXVCRKLGG

SEQ ID NO: 137 - protein, 20 aa, Artificial Sequence
  Description of Artificial Sequence Synthetic peptide
GGLYSCHFGKLTXVCRKLGG

SEQ ID NO: 138 - protein, 20 aa, Artificial Sequence
  Description of Artificial Sequence Synthetic peptide
GGTYSCHFGKVTWVCKKQGG

SEQ ID NO: 139 - protein, 20 aa, Artificial Sequence
  Description of Artificial Sequence Synthetic peptide
GGTYSCHFGKMTWVCKKQGG

SEQ ID NO: 140 - protein, 20 aa, Artificial Sequence
  Description of Artificial Sequence Synthetic peptide
GGNYSCHFGKLTWVCKKQGG

SEQ ID NO: 141 - protein, 20 aa, Artificial Sequence
  Description of Artificial Sequence Synthetic peptide
GGLYSCHFGKITXVCRKQGG

SEQ ID NO: 142 - protein, 20 aa, Artificial Sequence
  Description of Artificial Sequence Synthetic peptide
GGTYTCHFGKLTWVCQKLGG

SEQ ID NO: 143 - protein, 20 aa, Artificial Sequence
  Description of Artificial Sequence Synthetic peptide
GGLYACHXGKLTWVCKKQGG

SEQ ID NO: 144 - protein, 20 aa, Artificial Sequence
  Description of Artificial Sequence Synthetic peptide
GGLYSCHFGKITXVCKKQGG

SEQ ID NO: 145 - protein, 20 aa, Artificial Sequence
  Description of Artificial Sequence Synthetic peptide
GGLYSCHFGKLTXVCRKQGG

SEQ ID NO: 146 - protein, 20 aa, Artificial Sequence
  Description of Artificial Sequence Synthetic peptide
GGAYSCHFGKLTWVCKKQGG

SEQ ID NO: 147 - protein, 20 aa, Artificial Sequence
  Description of Artificial Sequence Synthetic peptide
GGSYSCHFGKLTWVCKKQGG

SEQ ID NO: 148 - protein, 20 aa, Artificial Sequence
  Description of Artificial Sequence Synthetic peptide
GGTLSCDFGKLGWVCDKQEG

SEQ ID NO: 149 - protein, 20 aa, Artificial Sequence
  Description of Artificial Sequence Synthetic peptide
GGTYKCHFGKLTWVCKKQGG

SEQ ID NO: 150 - protein, 20 aa, Artificial Sequence
  Description of Artificial Sequence Synthetic peptide
GGTYACHFGKLTWVCQKQGG

SEQ ID NO: 151 - protein, 20 aa, Artificial Sequence
  Description of Artificial Sequence Synthetic peptide
GGLYACHFGKITWVCKKQGG

SEQ ID NO: 152 - protein, 20 aa, Artificial Sequence
  Description of Artificial Sequence Synthetic peptide
GGLYSCHFGKLTWVCQKLGG

SEQ ID NO: 153 - protein, 20 aa, Artificial Sequence
  Description of Artificial Sequence Synthetic peptide
GGTYACHFGKLTXVCKKLGG

SEQ ID NO: 154 - protein, 20 aa, Artificial Sequence
  Description of Artificial Sequence Synthetic peptide
GGLYACHFGKLTXVCRKQGG

SEQ ID NO: 155 - protein, 20 aa, Artificial Sequence
  Description of Artificial Sequence Synthetic peptide
GGTYSCHFGKLTWVCTKQGG

SEQ ID NO: 156 - protein, 20 aa, Artificial Sequence
  Description of Artificial Sequence Synthetic peptide
GGTYMCHFGKLTWVCKKQGG

SEQ ID NO: 157 - protein, 20 aa, Artificial Sequence
  Description of Artificial Sequence Synthetic peptide
GGTYACHFGKLTXVCKKQGG

SEQ ID NO: 158 - protein, 20 aa, Artificial Sequence
  Description of Artificial Sequence Synthetic peptide
GGTYSCWFGKLTWVCKKQGG

SEQ ID NO: 159 - protein, 20 aa, Artificial Sequence
  Description of Artificial Sequence Synthetic peptide
GGTLSCDFGGLGWVCDKQEG

SEQ ID NO: 160 - protein, 20 aa, Artificial Sequence
  Description of Artificial Sequence Synthetic peptide
GGLYSCHMGKLTXVCRKQGG

SEQ ID NO: 161 - protein, 20 aa, Artificial Sequence
  Description of Artificial Sequence Synthetic peptide
GGLYSCHFGKLTXVCKKQRG

SEQ ID NO: 162 - protein, 20 aa, Artificial Sequence
  Description of Artificial Sequence Synthetic peptide
GGLYSCHMGKLTXECKKQGG

SEQ ID NO: 163 - protein, 20 aa, Artificial Sequence
  Description of Artificial Sequence Synthetic peptide
GGTYTCHFGKLTWVCQKQGG

SEQ ID NO: 164 - protein, 20 aa, Artificial Sequence
  Description of Artificial Sequence Synthetic peptide
GGLYACHMGKLTWVCKKQGG

SEQ ID NO: 165 - protein, 20 aa, Artificial Sequence
  Description of Artificial Sequence Synthetic peptide
GGTYSCHFGKLTXVCKKQGG

SEQ ID NO: 166 - protein, 20 aa, Artificial Sequence
  Description of Artificial Sequence Synthetic peptide
GGLYSCHMGKLTXVCKKQGG

SEQ ID NO: 167 - protein, 20 aa, Artificial Sequence
  Description of Artificial Sequence Synthetic peptide
GGLYACHFGKLTWVCKKQGG

SEQ ID NO: 168 - protein, 20 aa, Artificial Sequence
  Description of Artificial Sequence Synthetic peptide
GGLYSCHFGKITWVCRKQRG

SEQ ID NO: 169 - protein, 20 aa, Artificial Sequence
  Description of Artificial Sequence Synthetic peptide
GGTYSCHFGKLTWECKKQGG

SEQ ID NO: 170 - protein, 20 aa, Artificial Sequence
  Description of Artificial Sequence Synthetic peptide
GGTYSCLFGKLTWVCKKQGG

SEQ ID NO: 171 - protein, 20 aa, Artificial Sequence
  Description of Artificial Sequence Synthetic peptide
GGTYSCHFGKLTWVCKKQGG

SEQ ID NO: 172 - protein, 20 aa, Artificial Sequence
  Description of Artificial Sequence Synthetic peptide
GGTYSCHFGKLTWVCRKQGG

SEQ ID NO: 173 - protein, 20 aa, Artificial Sequence
  Description of Artificial Sequence Synthetic peptide
GGTYSCHXGKLTWVCKKQGG

SEQ ID NO: 174 - protein, 20 aa, Artificial Sequence
  Description of Artificial Sequence Synthetic peptide
GGTYSCHFGKLTWVCQKLGG

SEQ ID NO: 175 - protein, 20 aa, Artificial Sequence
  Description of Artificial Sequence Synthetic peptide
GGLYSCHFGKLTXVCKKQGG

SEQ ID NO: 176 - protein, 20 aa, Artificial Sequence
  Description of Artificial Sequence Synthetic peptide
GGLYSCHFGKITXVCRKQGG

SEQ ID NO: 177 - protein, 20 aa, Artificial Sequence
  Description of Artificial Sequence Synthetic peptide
GGTYTCHMGKLTXVCKKQGG

SEQ ID NO: 178 - protein, 20 aa, Artificial Sequence
  Description of Artificial Sequence Synthetic peptide
GGLYSCHFGKLTXVCRKQRG

SEQ ID NO: 179 - protein, 20 aa, Artificial Sequence
  Description of Artificial Sequence Synthetic peptide
GGTYTCHFGKLTXVCQKQGG

SEQ ID NO: 180 - protein, 20 aa, Artificial Sequence
  Description of Artificial Sequence Synthetic peptide
GGTYSCHFGKLTXVCQKQGG

SEQ ID NO: 181 - protein, 20 aa, Artificial Sequence
  Description of Artificial Sequence Synthetic peptide
GGTYTCHXGKLTWVCKKQGG

SEQ ID NO: 182 - protein, 20 aa, Artificial Sequence
  Description of Artificial Sequence Synthetic peptide
GGLYSCHMGKITWVCQKQRG

SEQ ID NO: 183 - protein, 20 aa, Artificial Sequence
  Description of Artificial Sequence Synthetic peptide
GGTYSCHFGKITWVCQKQGG

SEQ ID NO: 184 - protein, 20 aa, Artificial Sequence
  Description of Artificial Sequence Synthetic peptide
GGTYSCHFGKLTXVCKKQGG

SEQ ID NO: 185 - protein, 20 aa, Artificial Sequence
  Description of Artificial Sequence Synthetic peptide
GGTYSCHFGKLTWVCKKQRG

SEQ ID NO: 186 - protein, 20 aa, Artificial Sequence
  Description of Artificial Sequence Synthetic peptide
GGTYTCHFGKLTXVCKKQRG

SEQ ID NO: 187 - protein, 20 aa, Artificial Sequence
  Description of Artificial Sequence Synthetic peptide
GGLYSCHFGKLTWVCKKLGG

SEQ ID NO: 188 - protein, 20 aa, Artificial Sequence
  Description of Artificial Sequence Synthetic peptide
GGTYACHFGKLTWVCKKQRG

SEQ ID NO: 189 - protein, 20 aa, Artificial Sequence
  Description of Artificial Sequence Synthetic peptide
GGEYSCHFGKLTWVCKKQGG

SEQ ID NO: 190 - protein, 20 aa, Artificial Sequence
  Description of Artificial Sequence Synthetic peptide
GGLYSCHFGKLTWVCQKQRG

SEQ ID NO: 191 - protein, 20 aa, Artificial Sequence
  Description of Artificial Sequence Synthetic peptide
GGTYTCHFGKLTWVCRKQRG

SEQ ID NO: 192 - protein, 20 aa, Artificial Sequence
  Description of Artificial Sequence Synthetic peptide
GGTYSCHFGKATWVCKKQGG

SEQ ID NO: 193 - protein, 20 aa, Artificial Sequence
  Description of Artificial Sequence Synthetic peptide
GGLYACHFGKLTWICKKQRG

SEQ ID NO: 194 - protein, 20 aa, Artificial Sequence
  Description of Artificial Sequence Synthetic peptide
GGTYSCHFGKTTWVCKKQGG

SEQ ID NO: 195 - protein, 20 aa, Artificial Sequence
  Description of Artificial Sequence Synthetic peptide
GGTGSCEFGGLGWVCDKQGG

SEQ ID NO: 196 - protein, 20 aa, Artificial Sequence
  Description of Artificial Sequence Synthetic peptide
GGTYSCHFGKLTWVCKKQGG

SEQ ID NO: 197 - protein, 20 aa, Artificial Sequence
  Description of Artificial Sequence Synthetic peptide
GGTYTCHFGKLTXVCKKQRG

SEQ ID NO: 198 - protein, 20 aa, Artificial Sequence
  Description of Artificial Sequence Synthetic peptide
GGTGSCEFGKLTWVCDKQGG

SEQ ID NO: 199 - protein, 20 aa, Artificial Sequence
  Description of Artificial Sequence Synthetic peptide
GGTYSCHFGALTWVCKKQGG

SEQ ID NO: 200 - protein, 20 aa, Artificial Sequence
  Description of Artificial Sequence Synthetic peptide
GGTYSCHFGKLTWVCKKQGG

SEQ ID NO: 201 - protein, 20 aa, Artificial Sequence
  Description of Artificial Sequence Synthetic peptide
GGTYSCHFGPLTWVCKKQGG

SEQ ID NO: 202 - protein, 20 aa, Artificial Sequence
  Description of Artificial Sequence Synthetic peptide
GGTYSCHFGKLTWVCKPQGG

SEQ ID NO: 203 - protein, 19 aa, Artificial Sequence
  Description of Artificial Sequence Synthetic peptide
GGTYSCHFXLTWVCKPQGG

SEQ ID NO: 204 - protein, 19 aa, Artificial Sequence
  Description of Artificial Sequence Synthetic peptide
GGTYSCHFXLTWVCKKQGG

SEQ ID NO: 205 - protein, 20 aa, Artificial Sequence
  Description of Artificial Sequence Synthetic peptide
GGTYSCHFGKLTWVCKKQGG

SEQ ID NO: 206 - protein, 20 aa, Artificial Sequence
  Description of Artificial Sequence Synthetic peptide
GGTYSCHFGKLTWVCKPQGG

SEQ ID NO: 207 - protein, 20 aa, Artificial Sequence
  Description of Artificial Sequence Synthetic peptide
GGTYSCHFGRLTWVCKPQGG

SEQ ID NO: 208 - protein, 20 aa, Artificial Sequence
  Description of Artificial Sequence Synthetic peptide
GGTYSCHFGRLTWVCKKQGG

SEQ ID NO: 209 - protein, 19 aa, Artificial Sequence
  Description of Artificial Sequence Synthetic peptide
GGTYSCHFXLTWVCKPQGG

SEQ ID NO: 210 - protein, 19 aa, Artificial Sequence
  Description of Artificial Sequence Synthetic peptide
GGTYSCHFXLTWVCKKQGG

SEQ ID NO: 211 - protein, 20 aa, Artificial Sequence
  Description of Artificial Sequence Synthetic peptide
GGTYSCHFGKLTXVCKKQRG

SEQ ID NO: 212 - protein, 40 aa, Artificial Sequence
  Description of Artificial Sequence Synthetic peptide
GGTYSCHFGKLTWVCKKQGGGGTYSCHFGKLTWVCKKQGG

SEQ ID NO: 213 - protein, 40 aa, Artificial Sequence; identical to SEQ ID NO: 212
  Description of Artificial Sequence Synthetic peptide

SEQ ID NO: 214 - protein, 39 aa, Artificial Sequence
  Description of Artificial Sequence Synthetic peptide
GGTYSCHFGKLTWVCKKQGGGTYSCHFGKLTWVCKKQGG

SEQ ID NO: 215 - protein, 19 aa, Artificial Sequence
  Description of Artificial Sequence Synthetic peptide
GGTYSCHFGKLTWVCKKQG

SEQ ID NO: 216 - protein, 40 aa, Artificial Sequence
  Description of Artificial Sequence Synthetic peptide
GGTYSCHFGKLTWVCKKKGGGGTYSCHFGKLTWVCKKDGG

SEQ ID NO: 217 - protein, 21 aa, Artificial Sequence
  Description of Artificial Sequence Synthetic peptide
GGTYSCHFGKLTWVCKKKKGG

SEQ ID NO: 218 - protein, 21 aa, Artificial Sequence
  Description of Artificial Sequence Synthetic peptide
GGTYSCHFGKLTWVCKKKDGG

SEQ ID NO: 219 - protein, 40 aa, Artificial Sequence
  Description of Artificial Sequence Synthetic peptide
GGTYSCHFGKLTXVCKKQRGGGTYSCHFGKLTXVCKKQRG

SEQ ID NO: 220 - protein, 41 aa, Artificial Sequence
  Description of Artificial Sequence Synthetic peptide
CGGTYSCHFGKLTWVCKKQGGGGTYSCHFGKLTWVCKKQGG

SEQ ID NO: 221 - protein, 41 aa, Artificial Sequence
  Description of Artificial Sequence Synthetic peptide
CGGTYSCHFGKLTXVCKKQRGGGTYSCHFGKLTXVCKKQRG

SEQ ID NO: 222 - protein, 20 aa, Artificial Sequence
  Description of Artificial Sequence Synthetic peptide
GGTYSCSFGKLTWVCKXQGG

SEQ ID NO: 223 - protein, 20 aa, Artificial Sequence
  Description of Artificial Sequence Synthetic peptide
GGTYSCHFGXLTWVCKXQGG

SEQ ID NO: 224 - protein, 22 aa, Artificial Sequence
  Description of Artificial Sequence Synthetic peptide
GGTYSCHFGKLTWVCKKQGGGG

SEQ ID NO: 225 - protein, 21 aa, Artificial Sequence
  Description of Artificial Sequence Synthetic peptide
GGTYSCHFGKLTWVCKKQGGG

SEQ ID NO: 226 - protein, 21 aa, Artificial Sequence
  Description of Artificial Sequence Synthetic peptide
GGTYSCHFGKLTWVCKKQGXG

SEQ ID NO: 227 - protein, 21 aa, Artificial Sequence
  Description of Artificial Sequence Synthetic peptide
GGTYSCHFGKLTWVCKKQXGG

SEQ ID NO: 228 - protein, 21 aa, Artificial Sequence
  Description of Artificial Sequence Synthetic peptide
GGGTYSCHFGKLTWVCKKQGG

SEQ ID NO: 229 - protein, 20 aa, Artificial Sequence
  Description of Artificial Sequence Synthetic peptide
GGTYSCHFGPLTWVCKKQGG

SEQ ID NO: 230 - protein, 20 aa, Artificial Sequence
  Description of Artificial Sequence Synthetic peptide
GGTYSCHFAKLTWVCKKQGG

SEQ ID NO: 231 - protein, 20 aa, Artificial Sequence
  Description of Artificial Sequence Synthetic peptide
GGTYSCHFGGLTWVCKPQGG

SEQ ID NO: 232 - protein, 41 aa, Artificial Sequence; identical to SEQ ID NO: 221
  Description of Artificial Sequence Synthetic peptide

SEQ ID NO: 233 - protein, 20 aa, Artificial Sequence
  Description of Artificial Sequence Synthetic peptide
GGTYSCHFGKLTXVCKKQRG

SEQ ID NO: 234 - protein, 10 aa, Artificial Sequence
  Description of Artificial Sequence Synthetic peptide
CXXXXXTWXC

SEQ ID NO: 235 - protein, 20 aa, Artificial Sequence
  Description of Artificial Sequence Synthetic peptide
GGTYSCHFGPLTWVCKPQGG

SEQ ID NO: 236 - protein, 24 aa, Artificial Sequence
  Description of Artificial Sequence Synthetic peptide
VLPLYRCRMGRETWECMRAAGVTK

SEQ ID NO: 237 - protein, 20 aa, Artificial Sequence
  Description of Artificial Sequence Synthetic peptide
GGTYSCHFGKLTWVCKKQGX

SEQ ID NO: 238 - protein, 20 aa, Artificial Sequence
  Description of Artificial Sequence Synthetic peptide
GGTYSCHFGKLTWVCKKQXG

SEQ ID NO: 239 - protein, 20 aa, Artificial Sequence
  Description of Artificial Sequence Synthetic peptide
GGTYSCHFGKLTWVCKKXGG

SEQ ID NO: 240 - protein, 20 aa, Artificial Sequence
  Description of Artificial Sequence Synthetic peptide
GGTYSCHFGKLTWVCKKXGG

SEQ ID NO: 241 - protein, 10 aa, Artificial Sequence
  Description of Artificial Sequence Synthetic peptide
CXXXXXTWXC

SEQ ID NO: 242 - protein, 10 aa, Artificial Sequence
  Description of Artificial Sequence Synthetic peptide
CXXXXXTWXC

SEQ ID NO: 243 - protein, 20 aa, Artificial Sequence
  Description of Artificial Sequence Synthetic peptide
GGLYSCHFGKLTXVCKKQRG

SEQ ID NO: 244 - protein, 20 aa, Artificial Sequence
  Description of Artificial Sequence Synthetic peptide
GGTYSCHFGKLTWVCKKXGG

SEQ ID NO: 245 - protein, 20 aa, Artificial Sequence
  Description of Artificial Sequence Synthetic peptide
GGTYSCHFGKLTWVCKKXGG

(19) United States
(12) Patent Application Publication
(10) Pub. No.: US 2009/0118195 A1
(43) Pub. Date: May 7, 2009

(54) Molecules Which Promote Hematopoiesis

(76) Inventors: Hans-Georg Frank, Kerkrade (NL); Franz-Peter Bracht, Dussseldorf (DE); Udo Haberl, Baesweiler (DE); Andreas Rybka, Gelsenkirchen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/667,290

(22) PCT Filed: Nov. 10, 2005
(86) PCT No.: PCT/EP05/12075

§ 371 Date: Aug. 2, 2007

(30) Foreign Application Priority Data
Nov. 10, 2004 (DE) 10 2004 054 422.0
Dec. 14, 2004 (EP) 04029536.2
Jan. 25, 2005 (US) 11041207
Feb. 2, 2005 (EP) 05002113.8
Jun. 13, 2005 (EP) 05012637.4
Jun. 23, 2005 (EP) 05013594.6
Sep. 14, 2005 (EP) 05020035.1

(51) Int. Cl. A61K 38/08; C07K 7/06; C07K 7/08; A61P 7/00; C07H 21/00; C07K 14/00

(52) U.S. Cl. 514/15; 530/328; 530/326; 530/327; 530/324; 536/23.1

(57) ABSTRACT

The invention relates to supravalent peptide compounds depicting an enhanced efficacy. Supravalent compounds comprise several at least bivalent peptide units that bind to a receptor target and are connected to a large polymeric carrier unit.

The present invention relates to peptides as binding molecules for the erythropoietin receptor, methods for the preparation thereof, medicaments containing these peptides, and their use in selected indications, preferably for treatment of various forms of anemia and stroke.

The hormone erythropoietin (EPO) is a glycoprotein constituted by 165 amino acids and having four glycosylation sites. The four complex carbohydrate side chains comprise 40 percent of the entire molecular weight of about 35 kD. EPO is formed in the kidneys and from there migrates into the spleen and bone marrow, where it stimulates the production of erythrocytes. In chronic kidney diseases, reduced EPO production results in erythropenic anemia. With recombinant EPO, prepared by genetic engineering, anemias can be treated effectively. EPO improves dialysis patients' quality of life. Not only renal anemia, but also anemia in premature newborns, inflammation and tumor-associated anemias can be improved with recombinant EPO. By means of EPO, a high dosage chemotherapy can be performed more successfully in tumor patients. Similarly, EPO improves the recovery of cancer patients if administered within the scope of radiation therapy.

In the treatment with EPO, a problem exists in that the required dosage regimens are based on frequent or continuous intravenous or subcutaneous applications because the protein is decomposed relatively quickly in the body. Therefore, the evolution of recombinant EPO-derived molecules goes towards selectively modifying the glycoprotein, for example, by additional glycosylation or pegylation, in order to increase stability and thus biological half-life time.

Another important issue associated with the treatment with recombinant EPO is the danger, that patients develop antibodies to recombinant EPO during treatment. This is due to the fact, that recombinant EPO is not completely identical to endogenous EPO. Once antibody formation is induced, it can lead to antibodies, which compromise the activity of endogenous erythropoietin as well. It frequently increases the dosage of recombinant EPO needed for treatment. Especially if such antibodies compromise the activity of endogenous EPO, this effect can be interpreted as a treatment-induced autoimmune disease. It is especially undesired e.g. in case of dialysis patients undergoing renal transplantation after months or years of EPO-treatment. The antibodies then can compromise the activity of endogenous EPO produced by the transplant and thus compromise erythropoietic activity of the transplanted organ. Presently, it is an open question, whether the modifications introduced in recombinant EPO in order to increase biological half-life time will aggravate or improve this problem. Generally, it would be expected that extensive modifications and longer half-life time will aggravate this problematic property.

An alternative strategy is the preparation of synthetic peptides from amino acids which do not share sequence homology or structural relationship with erythropoietin. It was shown that peptides, unrelated to the sequence of EPO, which are significantly smaller than erythropoietin can act as agonists (Wrighton et al., 1996). The same authors showed that such peptides can be truncated to still active minimal peptides with length of 10 amino acids.

Synthetic peptides mimicking EPO's activity are subject of the international laid open WO96/40749. It discloses mimetic peptides of 10 to 40 amino acids of a distinct consensus preferably containing two prolines at the position commonly referred to as position 10 and 17, one of which is considered to be essential.

Thus to date, all small peptide-based agonists of the EPO receptor have had a structure which contains at least one proline, often two proline residues in defined positions, usually numbered as position 10 and 17, referenced to their position in the very active erythropoietin-mimetic peptide EMP1 (international laid open WO96/40749; Wrighton et al., 1996, Johnson et al, 1997):

GGTYSCHFGPLTWVCKPQGG.

These prolines are considered indispensable to the effectiveness of the peptides. For the proline at position 17, this has been substantiated by interactions with the receptor, while the proline at position 10 was thought to be necessary for the correct folding of the molecule (see also Wrighton et al. 1996, 1997). The correct folding, supported by the specific stereochemical properties of proline, is usually a necessary precondition of biological activity. Generally, proline is a structure-forming amino acid which is often involved—as in this case—in the formation of hairpin structures and beta turns. Due to this property, inter alia, it is a frequent point of attack for post-proline-specific endopeptidases which destroy proline-containing peptides/proteins. A number of endogenous peptide hormones (angiotensins I and II, urotensins, thyreoliberin, other liberins, etc.) are inactivated by such “single-hit” post-proline cleavage. Half-life time of proline-containing EPO-mimetic peptides is thus shortened by the activity of these frequent and active enzymes.

Such peptides can be produced chemically and do not need recombinant production, which is much more difficult to control and to yield products with defined quality and identity. Chemical production of peptides of such small size can also be competitive in terms of production costs. Moreover, chemical production allows defined introduction of molecular variations such as glycosylation, pegylation or any other defined modifications, which can have a known potency to increase biological half-life. However, so far there has been no approval of any therapy with existing EPO mimetic peptides.

Thus it is the objective of the present invention to provide alternative synthetic peptides which exhibit at least essential parts of the biological activity of the native EPO and thus provide alternative means for efficient therapeutic strategies, in particular for the treatment of anemia or stroke.

According to one aspect of the invention there is provided a peptide of at least 10 amino acids in length, capable of binding to the EPO receptor and comprising an agonist activity. The peptide thus depicts EPO mimetic properties. EPO mimetic peptides according to this invention do not comprise proline in the position commonly referred to as position 10 of EPO mimetic peptides, but a positively charged amino acid (for numbering please refer e.g. to Johnson et al, 1997 describing the ancestral sequence of EMP 1).

Said proline at position 10 is located in an amino acid motif which is characteristic for a certain folding structure, namely the beta-turn motif (please refer to Johnson, 1997). Said beta-turn structure forms upon receptor binding. The EPO mimetic peptides according to the invention thus do not comprise a proline in the beta-turn motif at position 10 but a positively charged amino acid. Examples are K, R, H or respective non-natural amino acids such as e.g. homoarginine.

Furthermore, a peptide is provided which comprises the following sequence of amino acids:

X₆X₇X₈X₉X₁₀X₁₁X₁₂X₁₃X₁₄X₁₅

wherein each amino acid is selected from natural or unnatural amino acids and
X₆ is C, A, E, α-amino-γ-bromobutyric acid or homocysteine (hoc);

### X₇ is R, H, L, W or Y or S;

X₈ is M, F, I, homoserinmethylether (hsm) or norisoleucine;
X₉ is G or a conservative exchange of G;
X₁₀ is a non-conservative exchange of proline;
or X₉ and X₁₀ are substituted by a single amino acid;
X₁₁ is independently selected from any amino acid;

### X₁₂ is T or A;

X₁₃ is W, 1-nal, 2-nal, A or F;

### X₁₄ is D, E, I, L or V;

X₁₅ is C, A, K, α-amino-γ-bromobutyric acid or homocysteine (hoc) provided that either X₆ or X₁₅ is C or hoc.

The length of the described peptide consensus is preferably between ten to forty or fifty or sixty amino acids. Peptides of above sixty amino acids in length, even though technically suitable, are not necessarily preferred since with increasing length of the peptide synthesis is usually getting more complicated and thus costly. In preferred embodiments, the peptide consensus depicts a length of at least 10, 15, 18 or 20 amino acids. Of course they can be embedded respectively be comprised by longer sequences. The described peptide sequences can be perceived as binding domains for the EPO receptor. As EPO mimetic peptides they are capable of binding to the EPO receptor.

It was very surprising, that the peptides according to the invention do exhibit EPO mimetic activities although one or—according to some embodiments—even both prolines may be replaced by other natural or non-natural amino acids. In fact the peptides according to the invention have an activity comparable to that of proline-containing peptides. However, it is noteworthy that the amino acids substituting proline residues do not represent a conservative exchange but instead a non-conservative exchange. Preferably, a positively charged amino acid such as basic amino acids such as K, R and H and especially K is used for substitution. The non-conservative amino acid used for substitution can also be a non-natural amino acid and is preferably one with a positively charged side chain. Also comprised are respective analogues of the mentioned amino acids. A suitable example of a non-natural amino acid is homoarginine. According to one embodiment the peptide carries a positively charged amino acid in position 10 except for the natural amino acid arginine. According to this embodiment the proline 10 is thus substituted by an amino acid selected from K, H or a non-natural positively charged amino acid such as e.g. homoarginine. It is preferred that the peptides depict a lysine or homoarginine in position 10. As described above, also the proline in position 17 might be replaced by a non-conservative amino acid. In this respect it is also preferred, that said non-conservative amino acid is one with a positively charged side chain such as K, R, H or a respective non-natural amino acid such as e.g. homoarginine. According to a sub-embodiment of this embodiment the peptide carries a positively charged amino acid in position 17 except for the natural amino acid arginine. According to this embodiment the proline 17 is thus substituted by an amino acid selected from K, H or a non-natural positively charged amino acid such as homoarginine. It is preferred that the peptides depict a lysine or homoarginine in position 17.

Moreover, the sequences can have N-terminal and/or C-terminal acetylations and amidations. Some amino acids may also be phosphorylated.

According to the invention there is also provided a peptide that binds to the erythropoietin receptor and comprises a sequence of the following amino acids:

X₆X₇X₈X₉X₁₀X₁₁X₁₂X₁₃X₁₄X₁₅

wherein each amino acid is indicated by standard letter abbreviation and

### X₆ is C;

### X₇ is R, H, L or W;

### X₈ is M, F or I;

X₉ is G or a conservative exchange of G;
X₁₀ is a non-conservative exchange of proline;
X₁₁ is independently selected from any amino acid;

### X₁₂ is T;

### X₁₃ is W;

### X₁₄ is D, E, I, L or V;

### X₁₅ is C.

Furthermore, X₇ can be serine, X₈ can be hsm or norisoleucine and X₁₃ can also be 1-nal, 2-nal, A or F. The length of the peptide consensus is preferably between ten to forty or fifty or sixty amino acids. In preferred embodiments, the peptide consensus comprises at least 10, 15, 18 or 20 amino acids.

The peptides according to the invention may comprise besides L-amino acids or the stereoisomeric D-amino acids, unnatural/unconventional amino acids, such as e.g. alpha, alpha-disubstituted amino acids, N-alkyl amino acids or lactic acid, e.g. 1-naphthylalanine, 2-naphthylalanine, homoserine-methylether, β-alanine, 3-pyridylalanine, 4-hydroxyproline, O-phosphoserine, N-methylglycine (sarcosine), homoarginine, N-acetylserine, N-acetylglycine, N-formylmethionine, 3-methylhistidine, 5-hydroxylysine, nor-lysine, 5-aminolevulinic acid or aminovaleric acid. The use of N-methylglycine (MeG) and N-acetylglycine (AcG) is especially preferred, in particular in a terminal position. Also within the scope of the present invention are peptides which are retro, inverso and retro/inverso peptides of the defined peptides and those peptides consisting entirely of D-amino acids.

The present invention also relates to the derivatives of the peptides, e.g. oxidation products of methionine, or deamidated glutamine, arginine and C-terminus amide.

According to one embodiment of the invention the peptides do have a single amino acid substituting the amino acid residues X₉ and X₁₀. In this embodiment also both residues may be substituted by one non-natural amino acid, e.g. 5-aminolevulinic acid or aminovaleric acid.

In a further embodiment, the peptides according to the invention comprise the consensus sequence

X₄X₅X₆X₇X₈X₉X₁₀X₁₁X₁₂X₁₃X₁₄X₁₅

wherein X₆ to X₁₅ have the above meaning and wherein

### X₄ is Y;

X₅ is independently selected from any amino acid and is preferably A, H, K, L, M; S, T or I.

The peptides according to the invention may be extended and may comprise the consensus sequence

X₃X₄X₅X₆X₇X₈X₉X₁₀X₁₁X₁₂X₁₃X₁₄X₁₅X₁₆X₁₇X₁₈

wherein X₄ to X₁₅ have the above meaning and wherein
X₃ is independently selected from any amino acid, preferably D, E, L, N, S, T or V;
X₁₆ is independently selected from any amino acid, preferably G, K, L, Q, R, S or T, more preferred K, R, S or T;
X₁₇ is independently selected from any amino acid, preferably A, G, P, R, K, Y or a non-natural amino acid with a positively charged side chain, more preferred K or Har;
X₁₈ is independently selected from any amino acid.

In a further embodiment of the invention the peptides comprise X₆ as C, E, A or hoc, preferably C and/or X₇ as R, H or Y or S and/or X₈ as F or M and/or X₉ as G or A, preferably G and/or X₁₀ as K or Har and/or X₁₁ as V, L, I, M, E, A, T or norisoleucine and/or X₁₂ as T and/or X₁₃ as W and/or X₁₄ as D or V and/or X₁₅ as C or hoc, preferably C and/or X₁₇ as P, Y or A or a basic natural or non-natural amino acid. It is, however, also preferred that X₁₇ is K or a non-natural amino acid with a positively charged side chain such as e.g. homoarginine.

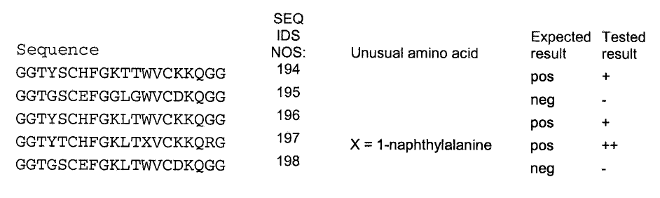

FIG. 19 discloses further novel and suitable peptide sequences depicting EPO mimetic activity. Further peptides depict the following sequences:

GGTYSCHFGALTWVCKKQGG
GGTYSCHFGKLTWVCKKQGG
GGTYSCHFGPLTWVCKKQGG
GGTYSCHFGKLWIVCKPQGG
GGTYSCHF-(ALS)-LTWVCKPQGG
GGTYSCHF-(ALS)-LTWVCKKQGG

With 5-aminolevulinic acid (5-Als):

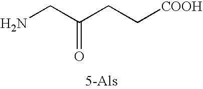

Also disclosed are peptides having a binding capacity to the receptor of the hormone erythropoietin and depicting an agonist activity which are characterised in that the peptides do not depict a proline. As described above, these peptides preferably do not comprise a proline in the positions commonly referred to as 10 and 17 but a different natural amino acid or 5-aminolevulinic acid. They preferably depict a lysine in position 17. Also disclosed are nucleic acids coding for respective peptides.

One or more conservative amino acid substitutions can be carried out within the amino acid sequence of the polypeptides according to this invention, wherein the substitution occurs within amino acids having unpolar side chains, the natural or non-natural uncharged D- or L amino acids with polar side chains, amino acids with aromatic side chains, the natural or non-natural positively charged D- or L-amino acids, the natural or non-natural negatively charged D- or L amino acids as well as within any amino acids of similar size and molecular weight, wherein the molecular weight of the original amino acid should not deviate more than approximately +/−25% of the molecular weight of the original amino acid and the binding capacity to the receptor of the hormone erythropoietin with agonistic effect is maintained. Preferably, no more than 1, 2 or 3 amino acids are substituted. Sequence variants wherein no proline is introduced at the positions 10 and 17 are preferred.

The peptide sequences described herein can be used as suitable monomeric peptide units which constitute binding domains for the EPO receptor. They can be used in there monomeric form since they bind to the EPO receptor. As described herein, they are preferably used as dimers since it was shown that the capacity to induce dimerisation of the EPO receptor and thus biological activity is enhanced by dimerisation of the monomeric binding units.

Thus it is clear that many different peptides are within the scope of the present invention. It has been found however, that the sequence Ac-VLPLYRCRMGRETWECMRAAGVTK-NH₂ has certain disadvantages and is thus not preferred according to the present invention.

At the beginning (N terminal) and end (C terminal) of the described individual peptide sequences, up to five amino acids may be removed and/or added. It is self-evident that size is not of relevance as long as the peptide function is preserved. Furthermore, please note that individual peptide sequences that might be too short to enfold their activity as monomers usually function as agonists upon dimerisation. Such peptides are thus preferably used in their dimeric form. Respective truncated and or elongated embodiments are thus also comprised by the spirit of the invention.

In the present invention, the abbreviations for the one-letter code as capital letters are those of the standard polypeptide nomenclature, extended by the addition of non-natural amino acids.

| Code | Amino acid |
|---|---|
| A | L-alanine |
| V | L-valine |
| L | L-leucine |
| I | L-isoleucine |
| M | L-methionine |
| F | L-phenylalanine |
| Y | L-tyrosine |
| W | L-tryptophan |
| H | L-histidine |
| S | L-serine |
| T | L-threonine |
| C | L-cysteine |
| N | L-asparagine |
| Q | L-glutamine |
| D | L-aspartic acid |
| E | L-glutamic acid |
| K | L-lysine |
| R | L-arginine |
| P | L-proline |
| G | glycine |
| Ava, 5-Ava | 5-aminovaleric acid |
| Als, 5-Als | 5-aminolevulinic acid |
| MeG | N-methylglycine |
| AcG | N-acetylglycine |
| Hsm | homoserine methylether |
| Har | homoarginine |
| 1nal | 1-naphthylalanine |
| 2nal | 2-naphthylalanine |
| βAla | beta-alanin |
| hoc | homocysteine |

As described above, the present invention also includes modifications of the peptides and defined peptide consensuses by, conservative exchanges of single amino acids. Such exchanges alter the structure and function of a binding molecule but only slightly in most cases. In a conservative exchange, one amino acid is replaced by another amino acid within a group with similar properties.

Examples of Corresponding Groups are
amino acids having non-polar side chains: A, G, V, L, 1, P, F, W, M
uncharged amino acids having polar side chains: S, T, G, C, Y, N, Q
amino acids having aromatic side chains: F, Y, W
positively charged amino acids: K, R, H
negatively charged amino acids: D, E
amino acids of similar size or molecular weight, wherein the molecular weight of the replacing amino acids deviates by a maximum of +/−25% (or +/−20%, +/−15%, +/−10%) from the molecular weight of the original amino acid.

It is self evident, that the groups also include non-natural amino acids with the respective side chain profile such as e.g. homoarginine in case of the group depicting positively charged side chains. In case a proline 10 substituting molecule such as e.g. a non-natural amino acid cannot be clearly assigned to one of the above groups characterized by their side-chain properties, it should usually be perceived as a non-conservative substitution of proline according to this invention. For categorizing these unusual amino acids, the classification aid according to the molecular weight might be helpful.

More specifically, Wrighton et al. (U.S. Pat. No. 5,773,569, and associated patents) examined in detail, using phage display techniques, which amino acids can be replaced, while maintaining the activity. They also investigated and published data on possible truncation, i.e. minimal length of a given EPO mimetic peptide. However, a proline near the central Gly-residue seemed to be the only possibility to obtain active peptides.

According to one embodiment of the invention there are provided peptides selected from the group consisting of SEQ ID NOS 2, 4-9 given below. Especially preferred is a peptide with a K in position 10 and a K in position 17 as is the case in SEQ ID NO 2.

SEQ ID NO 2: GGTYSCHFGKLTWVCKKQGG
SEQ ID NO 4: GGTYSCHFGKLTWVCKPQGG
SEQ ID NO 5: GGTYSCHFGRLTWVCKPQGG
SEQ ID NO 6: GGTYSCHFGRLTWVCKKQGG

Incorporation of 5-aminolevulinic acid (Als):

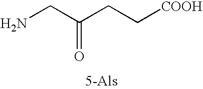

SEQ ID NO 7: GGTYSCHF-(Als)-LTWVCKPQGG
SEQ ID NO 8: GGTYSCHF-(Als)-LTWVCKKQGG
SEQ ID NO 9: GGTYSCHFGKLT-1nal-VCKKQRG

According to one embodiment peptide dimers or multimers are formed on the basis of the monomers according to SEQ ID NO 2 and 4 to 9 as given above or modifications thereof. The peptides described herein can e.g. also be modified by a conservative exchange of single amino acids, wherein preferably, not more than 1, 2 or 3 amino acids are exchanged.

Preferably these peptides are modified as to AcG at the N-terminus and MeG at the C-terminus.

As outlined above, the described peptides of the invention can be regarded as monomeric binding domains recognizing the binding site of the erythropoietin receptor. However, as was pointed out by Wrighton et al. (Wrighton 1997), two of these binding domains are generally needed in order to homodimerize the receptor and to induce signal transduction. Thus, it was not very surprising that a combination of two of these binding domains in one single molecule enhanced activity considerably, leading to the result that peptides with one single binding domain showed the same qualitative pattern of activity while two of the binding domains joint together show a much lower ED50 (Effect Dose 50%, a measure of activity). Peptides harboring two binding domains are specified as being bivalent or dimeric peptides within the context of this description and are particularly preferred.

One well-known technical solution for combining two monomeric binding domains is dimerization. All solutions following this approach are so far characterized by
a) the fact, that the binding domains are first synthesized separately as monovalent or monomeric peptides, which can be modified e.g. by attachment of reactive groups in preparation for step b
b) in a second reaction step, two—in most cases identical—binding domains are joined together in separate dimerization reaction, which can also include linker molecules usually being interposed between the two dimerised domains.

Such dimers are examples of bivalent peptides and exhibit essentially the same biological functions as the monomers. Usually, they show enhanced biological activity in case of EPO mimetic peptides.

Several techniques are known to the person skilled in the art to dimerize or oligomerize the monomers which can also be applied according to the teachings of the present invention. Monomers can be dimerized e.g. by covalent attachment to a linker. A linker is a joining molecule creating a covalent bond between the polypeptide units of the present invention. The polypeptide units can be combined via a linker in such a way, that the binding to the EPO receptor is: improved (Johnson et al. 1997; Wrighton et al. 1997). It is furthermore referred to the mulitimerization of monomeric biotinylated peptides by non-covalent interaction with a protein carrier molecule described by Wrighton et al (Wrighton, 1997). It is also possible to use a biotin/streptavdin system i.e. biotinylating the C-terminus of the peptides and a subsequent incubating the biotinylated peptides with streptavidin. Alternatively, it is known to achieve dimerization by forming a diketopiperazine structure. This method known to the skilled person is described in detail e.g. in Cavelier et al. (in: Peptides: The wave of the Future; Michal LebI and Richard A. Houghten (eds); American Peptide Society, 2001). The disclosure of these documents regarding the dimerization and a non-covalent multimerisation is incorporated herein by reference. Another alternative way to obtain peptide dimers known from prior art is to use bifunctional activated dicarboxylic acid derivatives as reactive precursors of the later linker moieties, which react with N-terminal amino groups, thereby forming the final dimeric peptide (Johnson et al, 1997). Monomers can also be dimerized by covalent attachment to a linker. Preferably the linker comprises NH—R—NH wherein R is a lower alkylene substituted with a functional group such as carboxyl group or amino group that enables binding to another molecule moiety. The linker might contain a lysine residue or lysine amide. Also PEG may be used a linker. The linker can be a molecule containing two carboxylic acids and optionally substituted at one or more atoms with a functional group such as an amine capable of being bound to one or more PEG molecules. A detailed description of possible steps for oligomerization and dimerization of peptides with a linking moiety is also given in WO 2004/101606.

The disclosure of these documents regarding the dimerization/multimerisation is incorporated herein by reference.

A peptide monomer or dimer May further comprise at least one spacer moiety. Preferably such spacer connects the linker of a monomer or dimer to a water soluble polymer moiety or a protecting group, which may be e.g. PEG. The PEG has a preferred molecular weight of at least 3 kD, preferably between 20 and 60 kD. The spacer may be a C1-12 moiety terminated with —NH-linkages or COOH-groups and optionally substituted at one or more available carbon atoms with a lower alkyl substituent. A particularly, preferred spacer is disclosed in WO 2004/100997. All documents—WO 2004/100997 and WO 2004/101606—are incorporated herein by reference. The PEG modification of peptides is disclosed in WO 2004/101600, which is also incorporated herein by reference.

Though being functionally sufficient and thus usable according to the teachings of the present invention, the prior art approaches of synthesizing dimeric molecules might have some disadvantages.

One potential drawback could be perceived in that the monomers to be connected have first to be synthesized separately. Because of the stochastic pairing of monomeric peptides during the dimerization respectively multimerisation reaction, it is in particular difficult to (selectively and intentionally) obtain heterodimeric bivalent/multivalent peptides with this approach. At least this would lead to great losses in yield of a special, intended heterodimer. Bi- or multivalent peptides harboring two or more slightly different monomeric binding domains are very desirable, since due to their heterodimeric nature, special interactions between the two domains, which are able to stabilize their interaction in the final bivalent peptide, can be introduced. However, due to the high losses in yield associated with the prior art “stochastic dimerization reactions”, this is usually economically not an attractive approach.

Applying the prior art approaches for dimerization—even though technically suitable—have thus some economic disadvantages for providing these peptides with heterogeneous binding domains as described. The invention, however, advantageously also teaches a much more efficient strategy to obtain highly active multi- or bivalent peptides, which even might contain heterogenous binding domains.

The core concept of this strategy refrains from synthesizing the monomeric peptides forming part of the multi- or bivalent peptide in separate reactions prior dimerization or multimerization, but to synthesize the final bi- or multivalent peptide in one step as a single peptide; e.g. in one single solid phase reaction. Thus a separate dimerization or multimerization step is no longer needed. This aspect provides a big advantage, i.e. the complete and independent control on each sequence position in the final peptide unit. The method allows to easily harbor at least two different receptor-specific binding domains in a peptide unit due to independent control on each sequence position.

According to this embodiment the sequence of the final peptide between the binding domains (which is the “linker region”) is composed of amino acids only, thus leading to one single, continuous bi- or multivalent EPO mimetic peptide. In a preferred embodiment of the invention the linker is composed of natural or unnatural amino acids which allow for a high conformational flexibility. In this regard it can be advantageous to use glycine residues as linking amino acids, which are known for their high flexibility in terms of torsions. However, also other amino acids, such as alanine or beta-alanine, or a mixture thereof can be used. The number and choice of used amino acids depend on the respective steric facts. This embodiment of the invention allows the custom-made design of a suitable linker by molecular modeling in order to avoid distortions of the bioactive conformation. A linker composed of 3 to 5 amino acids is especially preferred.

It is noteworthy that the linker between the functional domains (or monomeric units) of the final bivalent or multivalent peptides can be either a distinct part of the peptide or can be composed—fully or in parts—of amino acids which are part of the monomeric functional domains. For example the glycine residues in amino acid positions 1 and 2 and 19 and 20 can form part of the linker. Examples are given with Seq. 11 to 14. Thus the term “linker” is thus rather defined functionally than structurally, since an amino acid might form part of the linker unit as well as of the monomeric subunits.

Since—as mentioned above—during the synthesis of the bivalent/multivalent peptide each sequence position within the final peptide is under control and thus can be precisely determined it is possible to custom- or tailor make the peptides or specific regions or domains thereof, including the linker. This is of specific advantage since it allows the avoidance of distortion of the bioactive conformation of the final bivalent peptide due to unfavorable intramolecular interactions. The risk of distortions can be assessed prior to synthesis by molecular modeling. This especially applies to the design of the linker between the monomeric domains.

The continuous bivalent/multivalent peptides according to the invention show much higher activity then the corresponding monomeric peptides and thus confirm the observation known from other dimeric peptides that an increase of efficacy is associated with bivalent peptide concepts.

As for the monomers and dimeric peptides, the continuous bivalent/multivalent peptides can be modified by e.g. acetylation or amidation or be elongated at C-terminal or N-terminal positions. The prior art modifications for the monomeric peptides (monomers) mentioned above including the attachments of soluble moieties such as PEG, starch or dextrans are also applicable for the multi- or bivalent peptides according to the invention.

All possible modifications also apply for modifying the linker. In particular it might be advantageous to attach soluble polymer moieties to the linker such as e.g. PEG, starch or dextrans.

The synthesis of the final multi- or bivalent peptide according to the invention favorably can also include two subsequent and independent formations of disulfide bonds or other intramolecular bonds within each of the binding domains. Thereby the peptides can also be cyclized.

The bivalent structures according to the invention are favorably formed on the basis of the peptide monomers reported herein.

Some examples of appropriate peptide units for dimerising the EPO receptor are subsequently listed. The bars over the binding domains symbolize optional but preferred intramolecular disulfide bridges:

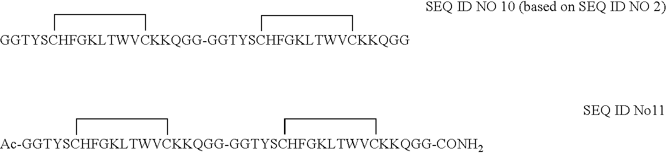

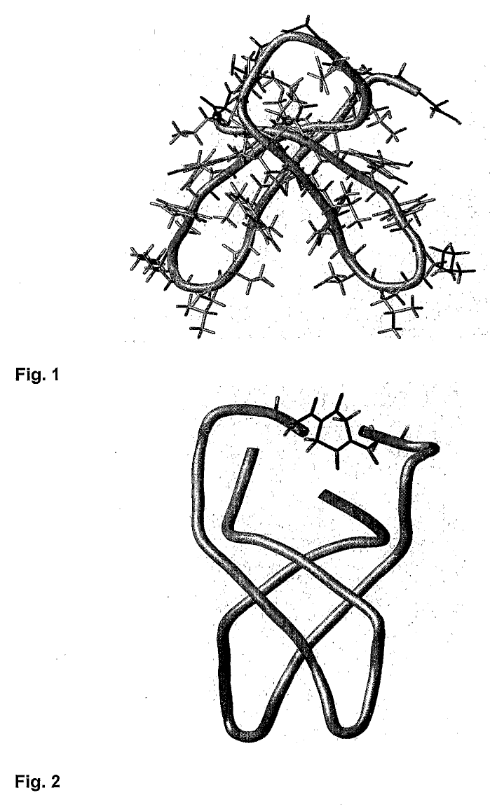

The linker in these bivalent structures is custom-made by molecular modelling to avoid distortions of the bioactive conformation (FIG. 1).

### SEQ ID NO 12

The linker sequence can be shortened by one glycine residue. This sequence is also an example for a linker composed by glycine residue forming at the same time part of the binding domain (see SEQ ID NO 2).

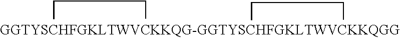

The binding domains can also be used as a monomer sequence (SEQ ID NO 13)

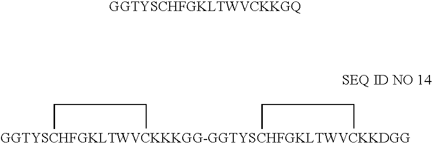

This sequence presents a continuous bivalent peptide according to the invention harboring two slightly different (heterogeneous) binding domains. Such bivalent peptides would not be accessible economically with a prior art dimerization approach (see above). Also these binding domains can be applied as a monomer as

SEQ ID NO 15:
GGTYSCHFGKLTWVCKKKKGG.
SEQ ID NO 15a:
GGTYSCHFGKLTWVCKKKDGG.

A further example is

GGTYSCHFGKLT-1nal-VCKKQRG-GGTYSCHFGKLT-1nal-
VCKKQRG

According to a further embodiment the peptide optionally carries an additional amino acid, preferably one with a reactive side chain such as cysteine at the N-terminus such as e.g. in the following sequences

C-GGTYSCHFGKLTWVCKKQGG-GGTYSCHFGKLTWVCKKQGG
C-GGTYSCHFGKLT-1nal-VCKKQRG-GGTYSCHFGKLT-1nal-
VCKKQRG

Further peptide examples depict the following amino acid sequence:

GGTYSCSFGKLTWVCK-Har-QGG
GGTYSCHFG-Har-LTWVCK-Har-QGG

The first sequence depicts a serine in position X₇. It was found that a new hydrogen bridge is created through the introduction of the hydroxyl group when this sequence is incorporated in a dimer. The use of a serine in position X₇ is thus especially favourable for dimers since the bioactive conformation is stabilised.

The second sequence depicting the non-natural amino acid homoarginine is especially suitable for use in a pharmaceutical composition for veterinary purposes. It was generally found that peptide sequences carrying an amino acid with a long positively charged side chain such as for example homoarginine in positions 10 and/or 17 depict a strong binding capacity to EPO receptors such as e.g. the mouse/dog receptor. They are thus especially suitable for use in veterinary products, however, their use is not limited thereto.

The reactive side chains may serve as a linking tie e.g. for further modifications.

The peptides furthermore optionally comprise intramolecular disulfide bridges between the first and second and/or third and fourth cysteine.

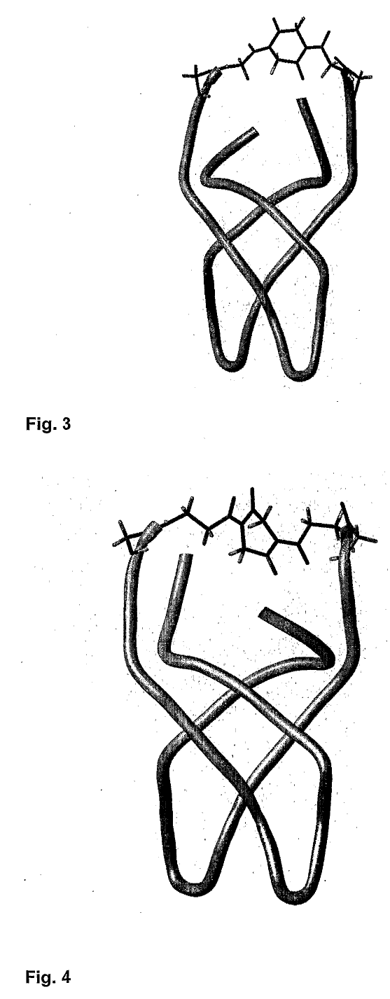

It is important to notice that the monomeric peptides as exemplified by SEQ ID 2, 4-9 and 12, 13 and 15, 15a are favorably combined to the continuous bivalent peptides according to the invention. However, also prior art methods for dimerization of these monomers can be applied. Examples for these prior art approaches being applied to the monomer peptides falling under the scope of this invention include (but are not limited to):
1. The dimerization via connection from C-terminus to C-terminus wherein the C-terminus of one of said monomeric peptide is covalently bound to the C-terminus of the other peptide. The linker/spacer between the monomers can contain a diketopiperazine unit. A preferred Gly-Gly diketopiperazine scaffold can be achieved by activating the C-terminal glycine monomer. This principle can also be use for forming a C-terminal dimerization.
   The following formulae and examples represent four customized examples which were optimized by molecular modeling:
   (a) dimer on the basis of SEQ ID NO 2 (the dimer conformation is showed in FIG. 2).

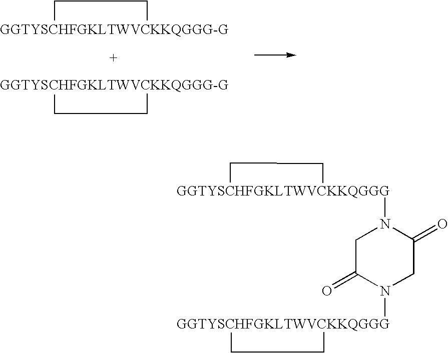

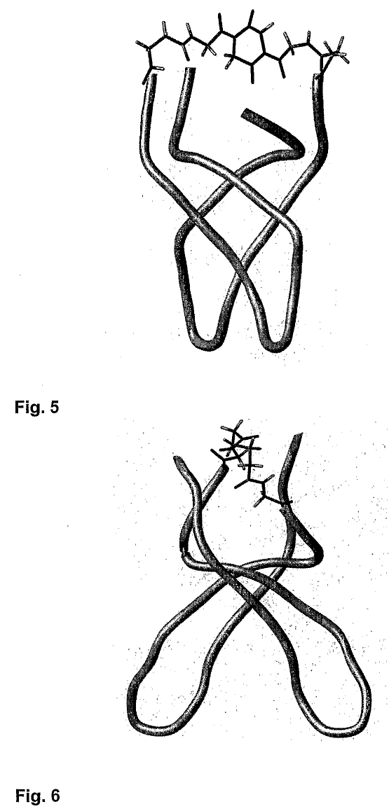

(b) dimer on the basis of SEQ ID NO 2 with a linker shortened by one glycine; the conformation is shown in FIG. 3.

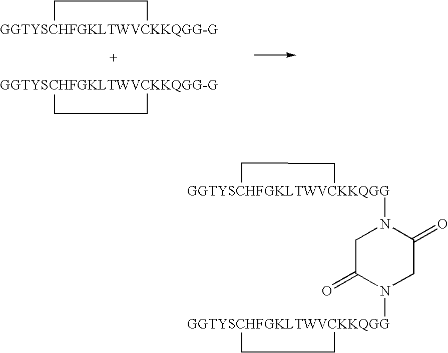

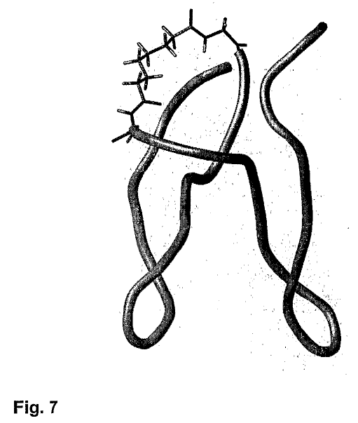

(c) dimer on the basis of SEQ ID NO 2 with a glycine substituted by beta-alanine (FIG. 4). The monomer (SEQ ID NO 16) is also applicable as EPO mimetic peptide.

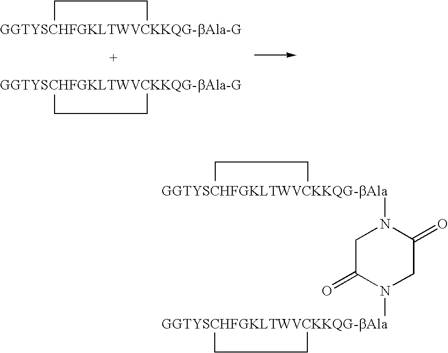

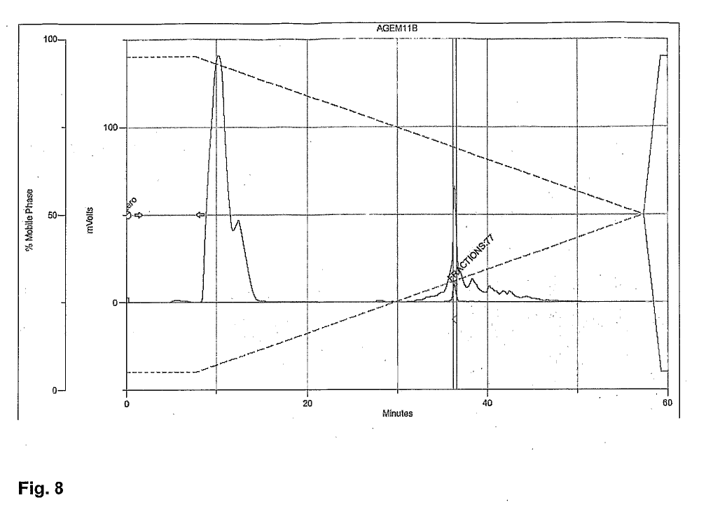

(d) dimer on the basis of SEQ ID NO 2 with an alternative glycine substituted by beta-alanine (FIG. 5). The monomer (SEQ ID NO 17) can also be applied as a EPO mimetic peptide.

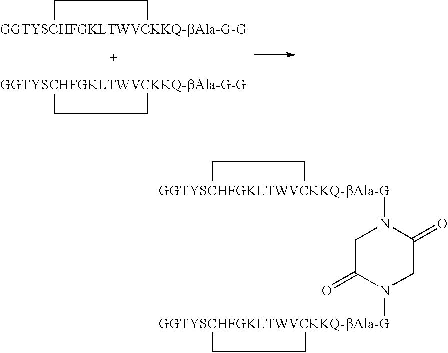

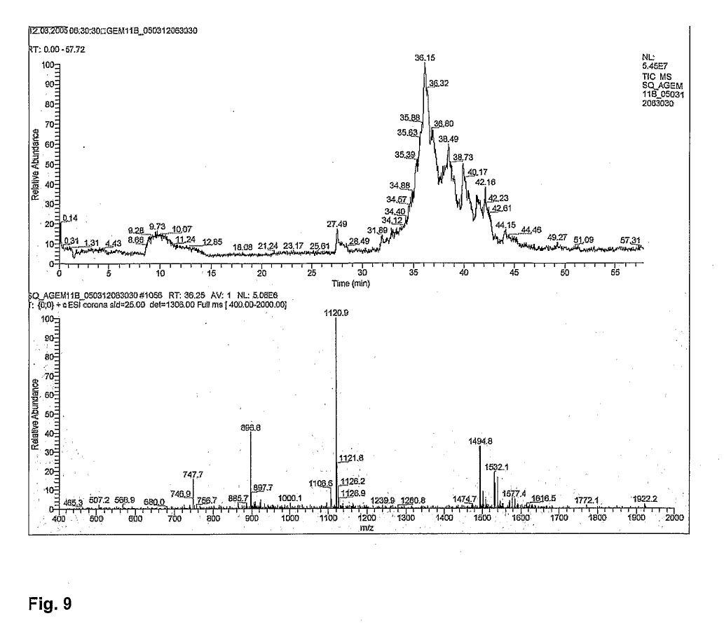

2. The dimerization via connection from N-terminus to N-terminus wherein the N-terminus of one of said monomeric peptides is covalently bound to the N-terminus of the other peptide, whereby the spacer unit is preferably containing a dicarboxylic acid building block.
   (a) In one embodiment the resulting dimers on the basis of SEQ ID NO 2 elongated at the N-Terminus by one glycine residue (SEQ ID NO 18) contain hexanedioyl unit as linker/spacer (FIG. 6):

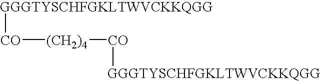

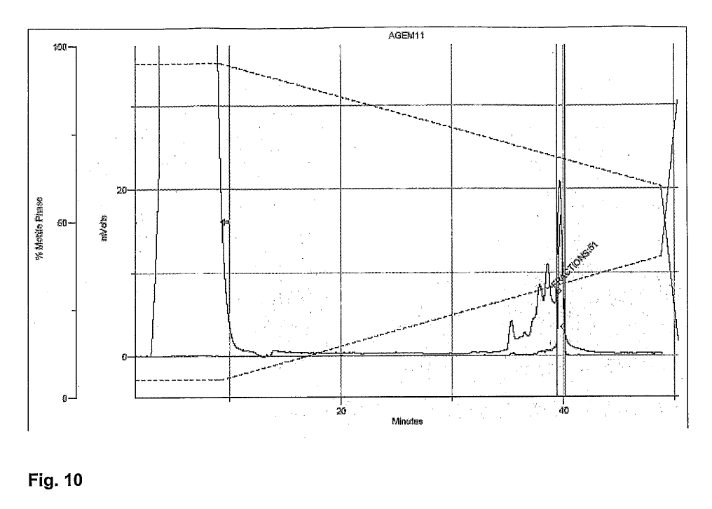

(b) In an alternative embodiment the dimerization can be achieved by using a octanedioyl unit as linker/spacer (FIG. 7):

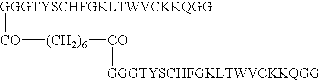

3. The dimerization via the side chains wherein an amino acid side chain of one of said monomeric peptides is covalently bound to an amino acid side chain of the other peptide with inclusion of a suitable spacer molecule connecting the two peptide monomers. This can include:
   (a) the connection via an amide bond.

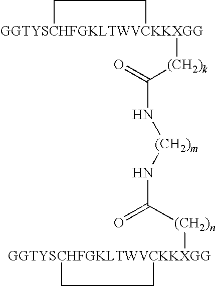

(b) or the connection via a disulfide bridge:

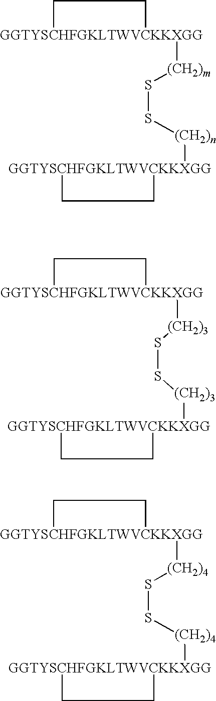

The X symbolizes the backbone core of the respective amino acid participating in the formation of the respective linking bond.

Respective assembly methods as described above can also be used for the preparation of multimers.

It is pointed out that all of the binding domains respectively peptides described herein either alone or as a part of a bivalent/multivalent peptide can also be used in a monomeric form and/or can be combined with one or more other either identical or different peptide domains in order to form respective homo- or heterogenous bi- or multivalent peptides.

The peptides can be modified by e.g. acetylation or amidation or be elongated at the C-terminal or N-terminal positions. Extension with one or more amino acids at one of the two termini, e.g. for preparation of an attachment site for a polymer often leads to a heterodimeric bivalent peptide unit which can best be manufactured as a continuous peptide.

The compounds of the present invention can advantageously be used for the preparation of human and/or veterinarian pharmaceutical compositions. As EPO mimetics they depict the basically the same qualitative activity pattern as erythropoietin. They are thus generally suitable for the same indications as erythropoietin.

Erythropoietin is a member of the cytokine superfamily. Besides the stimulating effects described in the introduction, it was also found that erythropoietin stimulates stem cells. The EPO mimetics described herein are thus suitable for all indications caused by stem cell associated effects. Non-limiting examples are the prevention and/or treatment of diseases associated with the nerve system. Examples are neurological injuries, diseases or disorders, such as e.g., Parkinsonism, Alzheimer's disease, Huntington's chorea, multiple sclerosis, amyotrophic lateral sclerosis, Gaucher's disease, Tay-Sachs disease, a neuropathy, peripheral nerve injury, a brain tumor, a brain injury, a spinal cord injury or a stroke injury. The EPO mimetic peptides according to the invention are also usable for the preventive and/or curative treatment of patients suffering from, or at risk of suffering from cardiac failure. Examples are cardiac infarction, coronary artery disease, myocarditis, chemotherapy treatment, alcoholism, cardiomyopathy, hypertension, valvar heart diseases including mitral insufficiency or aortic stenosis, and disorders of the thyroid gland, chronic and/or acute coronary syndrome.

Furthermore, the EPO mimetics can be used for stimulation of the physiological mobilization, proliferation and differentiation of endothelial precursor cells, for stimulation of vasculogenesis, for the treatment of diseases related to a dysfunction of endothelial precursor cells and for the production of pharmaceutical compositions for the treatment of such diseases and pharmaceutical compositions comprising said peptides and other agents suitable for stimulation of endothelial precursor cells. Examples of such diseases are hypercholesterolaemia, diabetis mellitus, endothel-mediated chronic inflammation diseases, endotheliosis including reticulo-endotheliosis, atherosclerosis, coronary heart disease, myocardic ischemia, angina pectoris, age-related cardiovascular diseases Raynaud disease, pregnancy induced hypertonia, chronic or acute renal failure, heart failure, wound healing and secondary diseases.

Furthermore, the peptides according to the invention are suitable carriers for delivering agents across the blood-brain barrier and can be used for respective purposes and/or the production of respective therapeutic conjugation agents capable of passing the blood-brain barrier.

The peptides described herein are especially suitable for the treatment of disorders that are characterized by a deficiency of erythropoietin or a low or defective red blood cell population and especially for the treatment of any type of anemia or stroke. The peptides are also suitable for increasing and/or maintaining hematocrit in a mammal. Such pharmaceutical compositions may optionally comprise pharmaceutical acceptable carriers in order to adopt the composition for the intended administration procedure. Suitable delivery methods as well as carriers and additives are for example described in WO 2004/101611 and WO 2004/100997.

As outlined above, dimerization of the monomeric peptides to dimers or even multimers usually improves the EPO mimetic agonist activity compared to the respective monomeric peptides. However, it is desirable to further enhance activity. For example, even dimeric EPO mimetic peptides are less potent than the EPO regarding the activation of the cellular mechanisms.

Several approaches were made in the prior art in order to increase the activity of the peptides, for example by variation of the amino acid sequence in order to identify more potent candidates. However, so far it is still desirable to further enhance the activity of peptides, especially of EPO mimetic peptides in order to improve the biological activity.

A further embodiment of the present invention provides a solution to that problem. Therein a compound is provided that binds target molecules and comprises
i) at least two peptide units wherein each peptide unit comprises at least two domains with a binding capacity to the target;
ii) at least one polymeric carrier unit;
   wherein said peptide units are bound to said polymeric carrier unit.

Surprisingly, it has been found that the combination of two or more bi- or multivalent peptides according to the invention on a polymeric support is greatly increasing the efficacy of the bivalent (or even multivalent) peptides to their binding receptor not only additively, but even over-additively. Thus a synergistic effect is observed.

The term “bivalent” as used for the purpose of the present invention is defined as a peptide comprising two domains with a binding capacity to a target, here in particular the EPO receptor. It is used interchangeably with the term “dimeric”. Accordingly, a “multivalent” or “multimeric” EPO mimetic peptide has several respective binding domains for the EPO receptor. It is self-evident that the terms “peptide” and “peptide unit” do not incorporate any restrictions regarding size and incorporate oligo- and polypeptides as well as proteins.

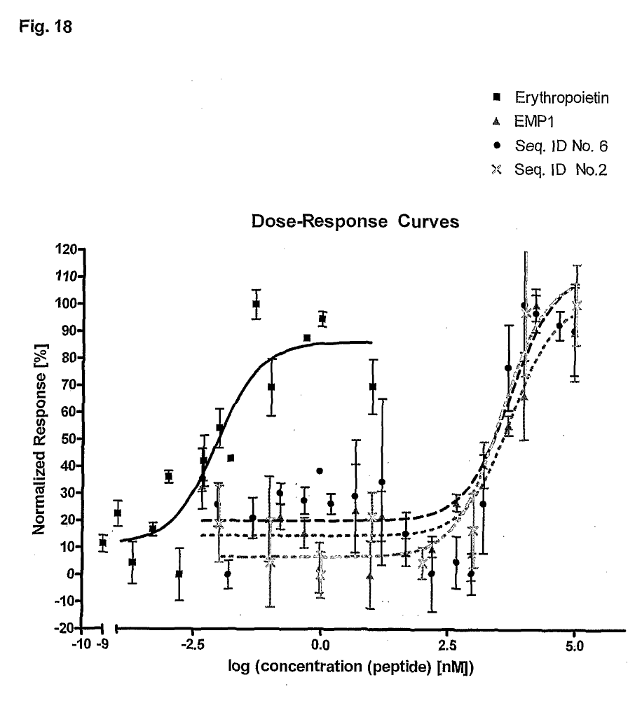

Compounds comprising two or more bi- or multivalent peptide units attached to a polymeric carrier unit are named “supravalent” in the context of this embodiment. These supravalent molecules greatly differ from the dimeric or multimeric molecules known in the state of the art. The state of the art combines merely monomeric EPO mimetic peptides in order to create a dimer. In contrast the supravalent molecules are generated by connecting already (at least) bivalent peptide units to a polymeric carrier unit thereby creating a supravalent molecule (examples are given in FIGS. 13 to 15). Thereby the overall activity and efficacy of the peptides is greatly enhanced thus decreasing the EC50 dose.

So far the reasons for the great potency of the supravalent molecules compared to the molecules known in the state of the art are not fully understood. It might be due to the fact that the dimeric molecules known in the state of the art provide merely one target respectively receptor binding unit per dimer. Thus only one receptor complex is generated upon binding of the dimeric compound thereby inducing only one signal transduction process. E.g. two monomeric EPO mimetic peptides are connected via PEG to form a peptide dimer thereby facilitating dimerisation of the receptor monomers necessary for signal transduction (Johnson et. al., 1997). In contrast, the supravalent compounds according to the invention comprise several already di- or multimeric respective receptor binding units. This might allow the generation of several receptor complexes on the cell surface per compound molecule thereby inducing several signal transductions and thereby potencing the activity of the peptide units over-additively. Binding of the supravalent compounds might result in a clustering of receptor complexes on the cell-surface.

The EPO mimetic peptide units used in this embodiment can be either homo- or heterogenic, meaning that either identical or differing peptide units are used. The same applies to the binding domains (monomeric peptides as described above) of the peptide units which can also be homo- or heterogenic. The bi- or multivalent peptide units bound to the carrier unit bind the same receptor target.

However, they can of course still differ in their amino acid sequence. The monomeric binding domains of the bi- or multivalent peptide units can be either linear or cyclic. A cyclic molecule can be for example created by the formation of intramolecular cysteine bridges (see above).

The polymeric carrier unit comprises at least one natural or synthetic branched, linear or dendritic polymer. The polymeric carrier unit is preferably soluble in water and body fluids and is preferably a pharmaceutically acceptable polymer. Water soluble polymer moieties include, but are not limited to, e.g. polyalkylene glycol and derivatives thereof, including PEG, PEG homopolymers, mPEG, polypropyleneglycol homopolymers, copolymers of ethylene glycol with propylene glycol, wherein said homopolymers and copoloymers are unsubstituted or substituted at one end e.g. with an acyl group; polyglycerines or polysialine acid; cellulose and cellulose derivatives, including methylcellulose and carboxymethylcellulose; starches (e.g. hydroxyalkyl starch (HAS), especially hydroxyethyl starch (HES) and dextrines, and derivatives thereof; dextran and dextran derivatives, including dextransulfat, crosslinked dextrin, and carboxymethyl dextrin; heparin and fragments of heparin; polyvinyl alcohol and polyvinyl ethyl ethers; polyvinylpyrrollidon; a,b-poly[(2-hydroxyethyl)-DL-aspartatamide; and polyoxyethylated polyols. Of course also other biologically inert water-soluble polymers can be used. A simple, but nevertheless preferred example of an appropriate carrier unit is a homobifunctional polymer, of for example polyethylene glycol (bis-maleimide, bis-carboxy, bis-amino etc.).

The polymeric carrier unit can have a wide range of molecular weight due to the different nature of the different polymers that are suitable in conjunction with the present invention. There are thus no size restrictions. However, it is preferred that the molecular weight is at least 3 kD, preferably at least 10 kD and approximately around 20 to 500 kD and more preferably around 30 to 150 or around 60 or 80 kD. The size of the carrier unit depends on the chosen polymer and can thus vary. For example, especially when starches such as hydroxyethylstarch are used, the molecular weight might be considerably higher. The average molecular weight might then be arranged around 100 to 4.000 kD or even be higher. The size of the carrier unit is preferably chosen such that each peptide unit is optimally arranged for binding their respective receptor molecules. In order to facilitate this, one embodiment of the present invention uses a carrier unit comprising a branching unit. According to this embodiment, the polymers, as for example PEG, are attached to a branching unit thus resulting in a large carrier molecule allowing the incorporation of numerous peptide units. Examples for appropriate branching units are glycerol or polyglycerol. Also dendritic branching units can be used as for example taught by Haag 2000, herein incorporated by reference.

Preferably, after the peptide units are created by combining the monomers (either head to head, head to tail, or tail to tail) the polymeric carrier unit is connected to the peptide units. The polymeric carrier unit is connected to the peptide units via a covalent or a non-covalent (e.g. a coordinative) bond. However the use of a covalent bond is preferred. The attachment can occur e.g. via a reactive amino acid of the peptide units e.g. lysine, cysteine, histidine, arginine, aspartic acid, glutamic acid, serine, threonine, tyrosine or the N-terminal amino group and the C-terminal carboxylic acid.

In case the polymeric carrier unit does not possess an appropriate coupling group, several coupling substances can be used in order to appropriately modify the polymer in order that it can react with at least one reactive group on the peptide unit. Suitable chemical groups that can be used to modify the polymer are e.g. as follows:

Acylating groups which react with the amino groups of the protein, for example acid anhydride groups, N-acylimidazole groups, azide groups, N-carboxy anhydride groups, diketene groups, dialkyl pyrocarbonate groups, imidoester groups, and carbodiimide-activated carboxyl-groups. All of the above groups are known to react with amino groups on proteins/peptides to form covalent bonds, involving acyl or similar linkages;

alkylating groups which react with sulfhydryl (mercapto), thiomethyl, imidazo or amino groups on the peptide unit, such as halo-carboxyl groups, maleimide groups, activated vinyl groups, ethylenimine groups, aryl halide groups, 2-hydroxy 5-nitro-benzyl bromide groups; and aliphatic aldehyde and ketone groups together with reducing agents, reacting with the amino group of the peptide;

ester and amide forming groups which react with a carboxyl group of the protein, such as diazocarboxylate groups, and carbodiimide and amine groups together;

disulfide forming groups which react with the sulfhydryl groups on the protein, such as 5,5′-dithiobis (2-nitrobenzoate) groups and alkylmercaptan groups (which react with the sulfhydryl groups of the protein in the presence of oxidizing agents such as iodine);

dicarbonyl groups, such as cyclohexandione groups, and other 1,2-diketone groups which react with the guanidine moieties of the peptide;

diazo groups, which react with phenolic groups on the peptide;

reactive groups from reaction of cyanogens bromide with the polysaccharide, which react with amino groups on the peptide.

Thus in summary, the compound according to the invention may be made by—optionally—first modifying the polymer chemically to produce a polymer having at least one chemical group thereon which is capable of reacting with an available or introduced chemical group on the peptide unit, and then reacting together the—optionally—modified polymer and the peptide unit to form a covalently bonded complex thereof utilising the chemical group of the—if necessary—modified polymer.

In case coupling occurs via a free SH-group of the peptide (e.g. of a cysteine group), the use of a maleimide group in the polymer is preferred.

In order to generate a defined molecule it is preferred to use a targeted approach for attaching the peptide units to the polymeric carrier unit. In case no appropriate amino acids are present at the desired attachment site, appropriate amino acids can be incorporated in the dimeric EPO mimetic peptide unit. For site specific polymer attachment a unique reactive group e.g. a specific amino acid at the end of the peptide, unit is preferred in order to avoid uncontrolled coupling reactions throughout the peptide leading to a heterogeneous mixture comprising a population of several different polyethylene glycol molecules.

The coupling of the peptide units to the polymeric carrier unit, e.g. PEG or HES, is performed using reactions principally known to the person skilled in the art. E.g. there are number of PEG and HES attachment methods available to those skilled in the art (see for example WO 2004/100997 giving further references, Roberts et al., 2002; U.S. Pat. No. 4,064,118; EP 1 398 322; EP 1 398 327; EP 1 398 328; WO 2004/024761; all herein incorporated by reference).

It is important to understand that the concept of supravalency described herein is different from the known concept of PEGylation or HESylation. In the state of the art e.g. PEGylation is only used in order to produce either peptide dimers or in order to improve pharmacokinetic parameters. However, as outlined above, the attachment of two or more at least bivalent peptide units to e.g. PEG as a polymeric carrier unit also greatly enhances efficacy (thus decreasing the EC50-dose). The concept of this invention thus has strong effects on pharmacodynamic parameters and not only on pharmacokinetic parameters as it is the case with the PEGylation concepts known in the state of the art. However, of course the incorporation of for example PEG as polymeric carrier unit also has the known advantages regarding pharmacokinetics:

PEGylation is usually undertaken to improve the biopharmaceutical properties of the peptides. The most relevant alterations of the protein molecule following PEG conjugation are size enlargement, protein surface and glycosylation function masking, charge modification and epitope shielding. In particular, size enlargement slows down kidney ultrafiltration and promotes the accumulation into permeable tissues by the passive enhance permeation and retention mechanism. Protein shielding reduces proteolysis and immune system recognition, which are important routes of elimination. The specific effect of PEGylation on protein physicochemical and biological properties is strictly determined by protein and polymer properties as well as by the adopted PEGylation strategy.

However, the use of PEG or other non-biodegradable polymers as support unit for a supravalent molecule can lead to new problems.

During in vivo applications, dosage intervals in a clinical setting are triggered by loss of effect of the drug. Routine dosages and dosage intervals are adapted such that the effect is not lost during dosage intervals. Due to the fact that peptides attached to a non-biodegradable, large polymer unit (e.g. a PEG-moiety) can be degraded faster than the support molecule might be eliminated by the body, a risk of accumulation of the carrier unit can arise. Such a risk of accumulation always occurs as effect-half life time of the drug is shorter than elimination half life time of the drug itself or one of its components/metabolites. Thus, accumulation of the carrier molecule should be avoided because peptides are usually PEGylated with very large PEG-moieties (˜20-40 kD) which thus show a slow renal elimination. The peptide moiety itself undergoes enzymatic degradation and even partial cleavage might suffice to deactivate the peptide.

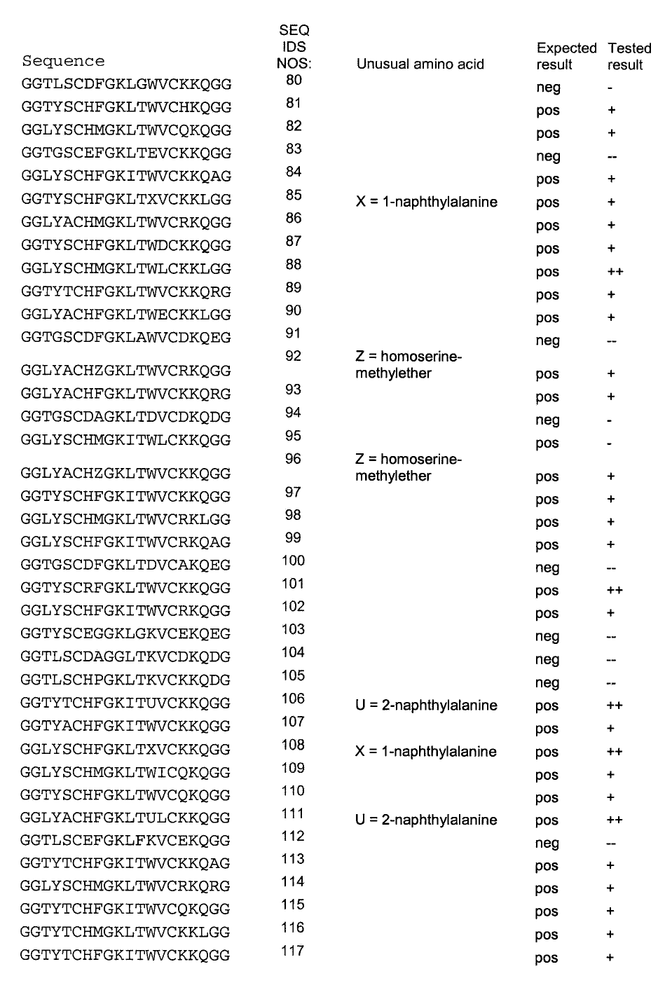

In order to find a solution to this problem one embodiment of the present invention teaches the use of a polymeric carrier unit that is composed of at least two subunits. The polymeric subunits are connected via biodegradable covalent linker structures. According to this embodiment the molecular weight of the large carrier molecule (for example 40 kD) is created by several small or intermediate sized subunits (for example each subunit having a molecular weight of 5 to 10 kD), that are connected via biodegradable linkers. The molecular weights of the modular subunits add up thereby generating the desired molecular weight of the carrier molecule. However, the biodegradable linker structures can be broken up in the body thereby releasing the smaller carrier subunits (e.g. 5 to 10 kD). The small carrier subunits show a better renal clearance than a polymer molecule having the overall molecular weight (e.g. 40 kD). An example is given in FIG. 16.

The linker structures are selected according to known degradation properties and time scales of degradation in body fluids. The breakable structures can, for instance, contain cleavable groups like carboxylic acid derivatives as amide/peptide bonds or esters which can be cleaved by hydrolysis (see e.g. Roberts, 2002 herein incorporated by reference). PEG succinimidyl esters can also be synthesized with various ester linkages in the PEG backbone to control the degradation rate at physiological pH (Zhao, 1997, herein incorporated by reference). Other breakable structures like disulfides of benzyl urethanes can be cleaved under mild reducing environments, such as in endosomal compartments of a cell (Zalipsky, 1999) and are thus also suitable. Other criteria for selection of appropriate linkers are the selection for fast (frequently enzymatic) degradation or slow (frequently non-enzymatic decomposition) degradation. Combination of these two mechanisms in body fluids is also feasible. It is clear that this highly advantageous concept is not limited to the specific peptide units described or referred to herein but also applies to other pharmaceutical molecules that are attached to large polymer units such as PEG molecules wherein the same problems of accumulation arises.

According to one embodiment hydroxyalkylstarch and preferably HES is used as polymeric carrier unit. HES has several important advantages. First of all, HES is biodegradable. Furthermore, the biodegradability of HES can be controlled via the ratio of ethyl groups and can thus be influenced. 30 to 50% ethyl groups are well suitable for the purpose of the present invention. Due to the biodegradability, accumulation problems as described above in conjunction with PEG do usually not occur. Furthermore, HES has been used for a long time in medical treatment e.g. in form of a plasma expander. Its innocuousness is thus approved.

Furthermore, derivatives of hydrolysis products of HES are detectable by gas chromatography. HES-peptide conjugates can be hydrolised under conditions under which the peptide units are still stable. This allows the quantification and monitoring of the degradation products and allows evaluations and standardisations of the active peptides.

According to a further embodiment a first type of polymeric carrier unit is used and loaded with peptide units. This first carrier is preferably easily biodegradable as is e.g. HES. However, not all attachment spots of the first carrier are occupied with peptide units but only e.g. around 20 to 50%. Depending on the size of the used polymer, several hundred peptide units can be coupled to the carrier molecule. The rest of the attachment spots of the first carrier are occupied with a different carrier, e.g. small PEG units having a lower molecular weight than the first carrier. This embodiment has the advantage that a supravalent composition is created due to the first carrier which is however, very durable due to the presence of the second carrier, which is constituted preferably by PEG units of 3 to 5 or 10 kD. However, the whole entity is very well degradable, since the first carrier (e.g. HES) and the peptide units are biodegradable and the second carrier, e.g. PEG is small enough to be easily cleared from the body.

The monomers constituting the binding domains of the peptide units recognize the homodimeric erythropoietin receptor. The latter property of being a homodimeric receptor differentiates the EPO-receptor from many other cytokine receptors. The peptide units comprising at least two EPO mimetic monomeric binding domains as described above bind the EPO receptor and preferably are able to di-respectively multimerise their target and/or stabilize it accordingly thereby creating a signal transduction inducing complex.

The present invention also comprises respective compound production methods, wherein the peptide units are connected to the respective carrier units. The present invention furthermore comprises respective compound production methods, wherein the peptide units are connected to the respective polymeric carrier units. The compounds of the present invention can advantageously be used for the preparation of human and/or veterinarian pharmaceutical compositions. They can be especially suitable for the treatment of disorders that are characterized by a deficiency of erythropoietin or a low or defective red blood cell population and especially for the treatment of any type of anemia and stroke. They are also usable for all indications described above. Such pharmaceutical compositions may optionally comprise pharmaceutical acceptable carriers in order to adopt the composition for the intended administration procedure. Suitable delivery methods as well as carriers and additives are for example described in WO 2004/100997 and WO 2004/101611, herein incorporated by reference.

### EXAMPLES

The concept of the supravalent molecules shall be explained by means of examples. FIG. 13 shows an example of a simple supravalent molecule according to the invention. Two continuous bivalent EPO mimetic peptides are connected N-terminally by a bifunctional PEG moiety carrying maleimide groups. Cysteine was chosen as reactive attachment site for the PEG carrier unit.

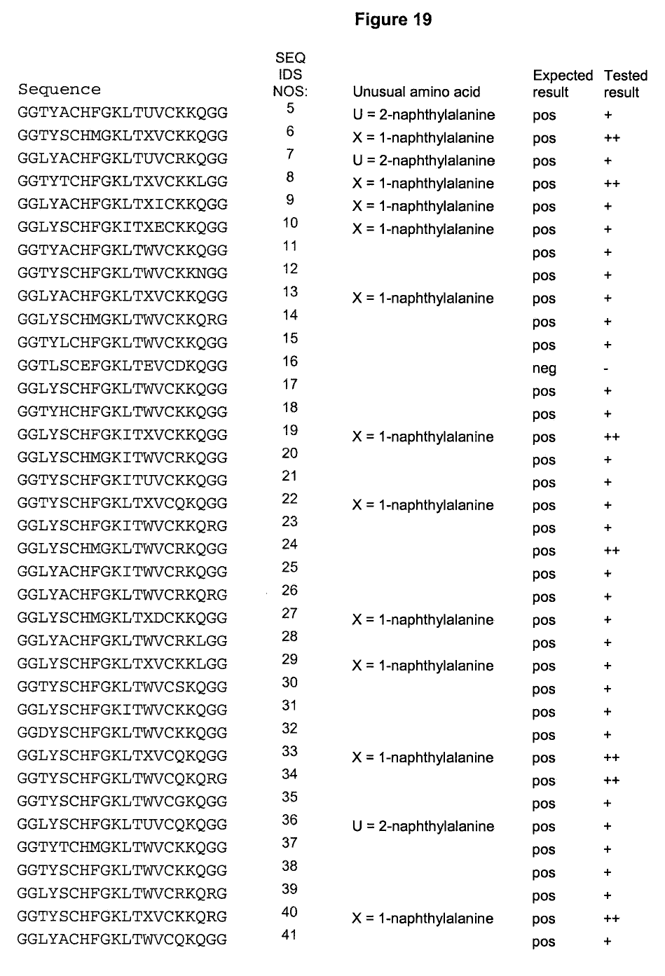

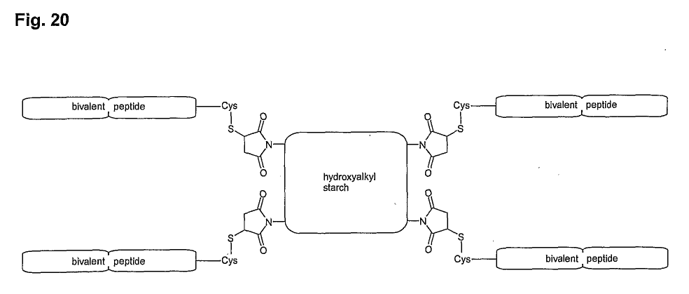

However, supravalent molecules can comprise more than two continuous bi- or multivalent peptide units. FIG. 14 gives an example that is based on a carrier unit with a central glycerol unit as branching unit and comprising three continuous bivalent peptides. Again cysteine was used for attachment. FIG. 20 shows an example using HES as polymeric carrier unit. HES was modified such that it carries maleimide groups reacting with the SH groups of the peptide units. According to the example, all attachment sites are bound to peptide units. However, also small PEG units (e.g. 3 to 10 kD) could occupy at least some of the attachment sites.

As explained above, the supravalent concept can also be extended to polyvalent dendritic polymers wherein a dendritic and/or polymer carrier unit is connected to a larger number of continuous bivalent peptides. For example, the dendritic branching unit can be based on polyglycerol (please refer to Haag 2000, herein incorporated by reference).

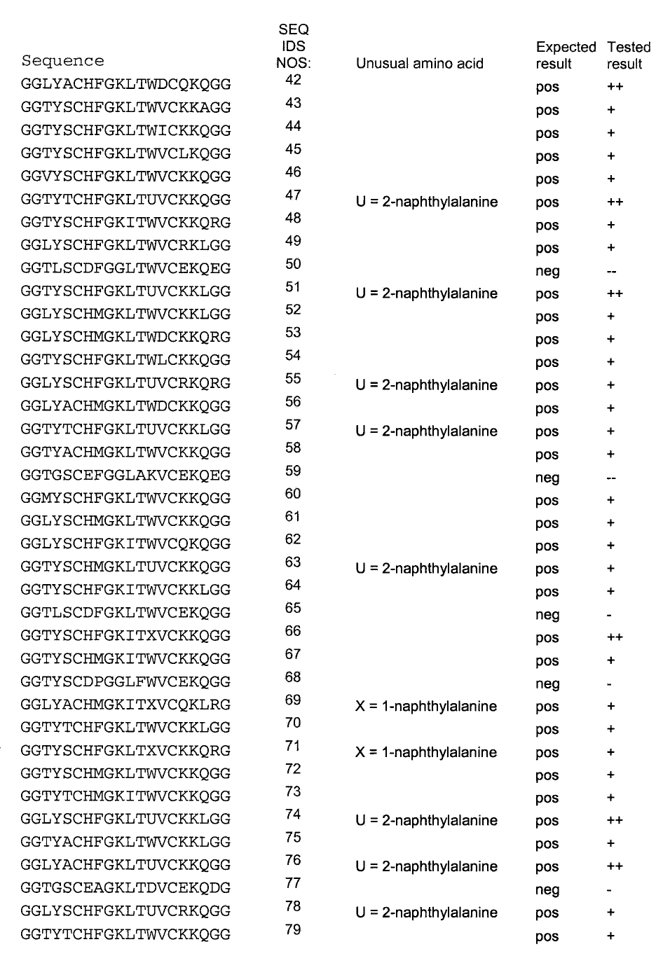

An example for a supravalent molecule based on a carrier unit with a dendritic branching unit containing six continuous bivalent peptides is shown in FIG. 15.

Other examples of supravalent molecules comprise carrier units with starches or dextrans, which are oxidized using e.g. periodic acid to harbor a large number of aldehyde functions. In a second step, many bivalent peptides are attached to the carrier unit and together form the final molecule. Please note that even several hundred (e.g. 50 to 1000, preferably 150 to 800, more preferably 250 to 700) peptide units can be coupled to the carrier molecule, which is e.g. HES.

FIG. 16 demonstrates the concept of a simple biodegradable supravalent molecule. Two continuous bivalent EPO mimetic peptides are connected N-terminally by two bifunctional PEG moieties that are connected via a biodegradable linker having an intermediate cleavage position. The linkers allow the break up of the large PEG unit in the subunits thereby facilitating renal clearance.

### I. Peptide Synthesis of Monomers

### Manual Synthesis

The synthesis is carried out by the use of a Discover microwave system (CEM) using PL-Rink-Amide-Resin (substitution rate 0.4 mmol/g) or preloaded Wang-Resins in a scale of 0.4 mmol. Removal of Fmoc-group is achieved by addition of 30 ml piperidine/DMF (1:3) and irradiation with 100 W for 3×30 sec. Coupling of amino acids is achieved by addition of 5 fold excess of amino acid in DMF PyBOP/HOBT/DIPEA as coupling additives and irradiation with 50 W for 5×30 sec. Between all irradiation cycles the solution is cooled manually with the help of an ice bath. After deprotection and coupling, the resin is washed 6 times with 30 ml DMF. After deprotection of the last amino acid some peptides are acetylated by incubation with 1.268 ml of capping solution (4.73 ml acetic anhydride and 8.73 ml DIEA in 100 ml DMSO) for 5 minutes. Before cleavage, the resin is then washed 6 times with 30 ml DMF and 6 times with 30 ml DCM. Cleavage of the crude peptides is achieved by treatment with 5 ml TFA/TIS/EDT/H₂O (94/1/2.5/2.5) for 120 minutes under inert atmosphere. This solution is filtered into 40 ml cold ether. The precipitate is dissolved in acetonitrile/water (1/1) and the peptide is purified by RP-HPLC (Kromasil 100 C18 10 μm, 250×4.6 mm).

### Automated Synthesis

The synthesis is carried out by the use of an Odyssey microwave system (CEM) using PL-Rink-Amide-Resins (substitution rate 0.4 mmol/g) or preloaded Wang-Resins in a scale of 0.25 mmol. Removal of Fmoc-groups is achieved by addition of 10 ml piperidine/DMF (1:3) and irradiation with 100 W for 10×10 sec. Coupling of amino acids is achieved by addition of 5 fold excess of amino acid in DMF PyBOP/HOBT/DIPEA as coupling additives and irradiation with 50 W for 5×30 sec. Between all irradiation cycles the solution is cooled by bubbling nitrogen through the reaction mixture. After deprotection and coupling, the resin is washed 6 times with 10 ml DMF. After deprotection of the last amino acid, some peptides are acetylated by incubation with 0.793 ml of capping-solution (4.73 ml acetic anhydride and 8.73 ml DIEA in 100 ml DMSO) for 5 minutes. Before cleavage the resin is then washed 6 times with 10 ml DMF and 6 times with 10 ml DCM. Cleavage of the crude peptides is achieved by treatment with 5 ml TFA/TIS/EDT/H₂O (94/1/2.5/2.5) for 120 minutes under an inert atmosphere. This solution is filtered into 40 ml cold ether, the precipitate dissolved in acetonitrile/water (1/1) and the peptide is purified by RP-HPLC (Kromasil 100 C18 10 μm, 250×4.6 mm).

### Purification

All peptides were purified using a Nebula-LCMS-system (Gilson). The crude material of all peptides was dissolved in acetonitrile/water (1/1) and the peptide purified by RP-HPLC (Kromasil 100 C18 10 μm, 250×4.6 mm). The flow rate was 20 ml/min and the LCMS split ratio 1/1000.

### II. Formation of the Intramolecular Disulfide Bridges

Cyclization with K₃[(FeCN₆)

Solution1: 10 mg of the peptide are dissolved in 0.1% TFA/acetonitrile and diluted with water until a concentration of 0.5 mg/ml is reached. Solid ammonium bicarbonate is added to reach a pH of app. 8.

Solution 2: In a second vial 10 ml 0.1% TFA/acetonitrile are diluted with 10 ml of water. Solid ammoniumbicarbonate is added until a pH of 8 is reached and 1 drop of a 0.1 M solution of K₃[(FeCN₆)] is added.

Solution 1 and 2 are added dropwise over a period of 3 hours to a mixture of acetonitrile/water (1/1; pH=8). The mixture is incubated at room temperature overnight and the mixture concentrated and purified by LCMS.

Cyclization with CLEAR-OX™-resin

To 100 ml of acetonitrile/water (1/1; 0.1% TFA), solid ammonium bicarbonate is added until a pH of 8 is reached. This solution is degassed by bubbling Argon for 30 minutes. Now 100 mg of CLEAR-OX™-resin is added. After 10 minutes, 10 mg of the peptide is added as a solid. After 2 h of incubation, the solution is filtered, concentrated and purified by LCMS.

### Purification of Cyclic Peptides:

All peptides were purified using a Nebula-LCMS-system (Gilson). The crude material of all peptides was dissolved in acetonitrile/water (1/1) or DMSO and the peptide was purified by RP-HPLC (Kromasil 100 C18 or C8 10 μm, 250×4.6 mm). The flow rate was 20 ml/min and the LCMS split ratio 1/1000.

III. In-Vitro Assays with Monomers

Proliferation assay with TF-1 cells by BrdU incorporation

TF-1 Cells in logarithmic growth phase (˜2.10⁵-1.10⁶ cells/ml; RPMI medium; 20% fetal calf serum; supplemented with Penicillin, streptomycin, L-Glutamine; 0.5 ng/ml Interleukin 3) are washed (centrifuge 5 min. 1500 rpm and resuspend in RPMI complete without IL3 at 500.000 cells/ml) and precultured before start of the assay for 24 h without IL-3. At the next day the cells are seeded in 24- or 96-well plates usually using at least 6 concentrations and 4 wells per concentration containing at least 10.000 cells/well per agent to be tested. Each experiment includes controls comprising recombinant EPO as a positive control agent and wells without addition of cytokine as negative control agent. Peptides and EPO-controls are prediluted in medium to the desired concentrations and added to the cells, starting a culture period of 3 days under standard culture conditions (37° C., 5% carbon dioxide in the gas phase, atmosphere saturated with water). Concentrations always refer to the final concentration of agent in the well during this 3-day culture period. At the end of this culture period, FdU is added to a final concentration of 8 ng/ml culture medium and the culture continued for 6 hours. Then, BrdU (bromodeoxyuridine) and dCd (2-deoxycytidine) are added to their final concentrations (10 ng/ml BrdU; 8 ng/ml dCD; final concentrations in culture medium) and culture continued for additional 2 hours.

At the end of this incubation and culture period, the cells are washed once in phosphate buffered saline containing 1.5% BSA and resuspended in a minimal amount liquid. From this suspension, cells are added dropwise into 70% ethanol at −20° C. From here, cells are either incubated for 10 min. on ice and then analysed directly or can be stored at 4° C. prior to analysis.

Prior to analysis, cells are pelleted by centrifugation, the supernatant is discarded and the cells resuspended in a minimal amount of remaining fluid. The cells are then suspended and incubated for 10 min. in 0.5 ml 2M HCl/0.5% triton X-100. Then, they are pelleted again and resuspended in a minimal amount of remaining fluid, which is diluted with 0.5 ml of 0.1N Na₂B₄O₇, pH 8.5 prior to immediate repelleting of the cells. Finally, the cells are resuspended in 40 μl of phosphate buffered saline (1.5% BSA) and divided into two reaction tubes containing 20 μl cell suspension each. 2 μl of anti-BrdU-FITC (DAKO, clone Bu20a) are added to one tube and 2 μl control mIgG1-FITC (Sigma) are added to the second tube starting an incubation period of 30 min. at room temperature. Then, 0.4 ml of phosphate buffered saline and 10 μg/ml Propidium Iodide (final concentration) are added. Analysis in the flow cytometer refers to the fraction of 4 C cells or cells with higher ploidy and to the fraction of BrdU-positive cells, thus determining the fraction of cells in the relevant stages of the cell cycle.

Proliferation assay with TF-1 cells by MTT

TF-1 Cells in logarithmic growth phase (˜2.10⁵-1.10⁶ cells/ml; RPMI medium; 20% fetal calf serum; supplemented with Penicillin, streptomycin, L-Glutamine; 0.5 ng/ml Interleukin 3) are washed (centrifuge 5 min. 1500 rpm and resuspend in RPMI complete without IL3 at 500.000 cells/ml) and precultured before start of the assay for 24 h without IL-3. At the next day the cells are seeded in 24- or 96-well plates usually using at least 6 concentrations and 4 wells per concentration containing at least 10.000 cells/well per agent to be tested. Each experiment includes controls comprising recombinant EPO as a positive control agent and wells without addition of cytokine as negative control agent. Peptides and EPO-controls are prediluted in medium to the desired concentrations and added to the cells, starting a culture period of 3 days under standard culture conditions (37° C., 5% carbon dioxide in the gas phase, atmosphere saturated with water). Concentrations always refer to the final concentration of agent in the well during this 4-day culture period.

At day 4, prior to start of the analysis, a dilution series of a known number of TF-1 cells is prepared in a number of wells (01250015000/10000120000150000 cells/well in 100 μl medium). These wells are treated in the same way as the test wells and later provide a calibration curve from which cell numbers can be determined. Having set up these reference wells, MTS and PMS from the MTT proliferation kit (Promega, CellTiter 96 Aqueous non-radioactive cell proliferation assay) are thawed in a 37° C. waterbath and 100 μl of PMS solution are added to 2 ml of MTS solution. 20 μl of this mixture are added to each well of the assay plates and incubated at 37° C. for 3-4 h. 25 μl of 10% sodium dodecylsulfate in water are added to each well prior to measurement E492 in an ELISA Reader.

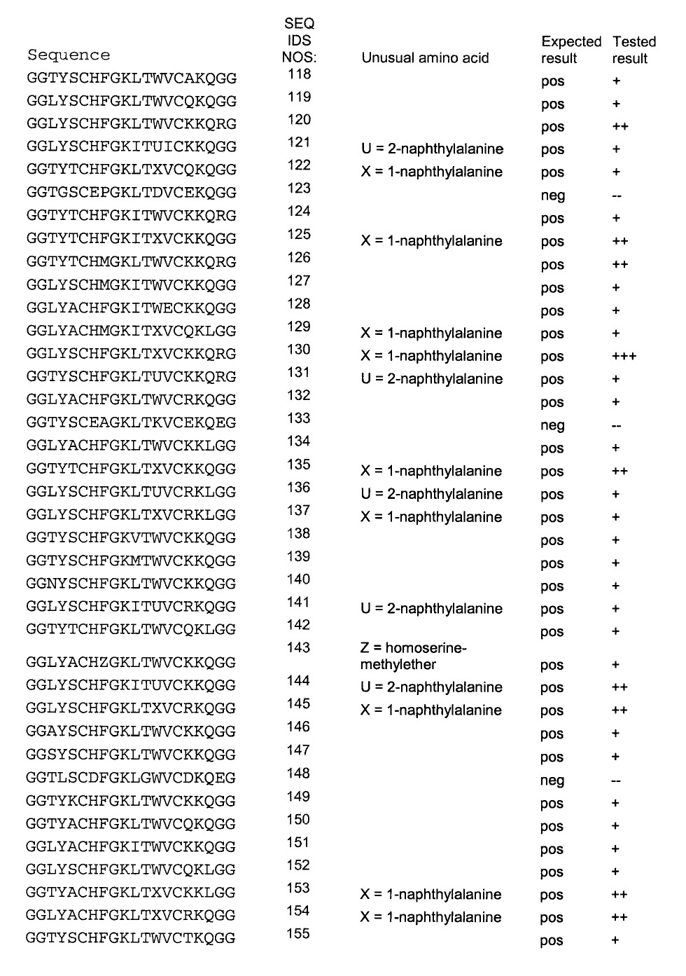

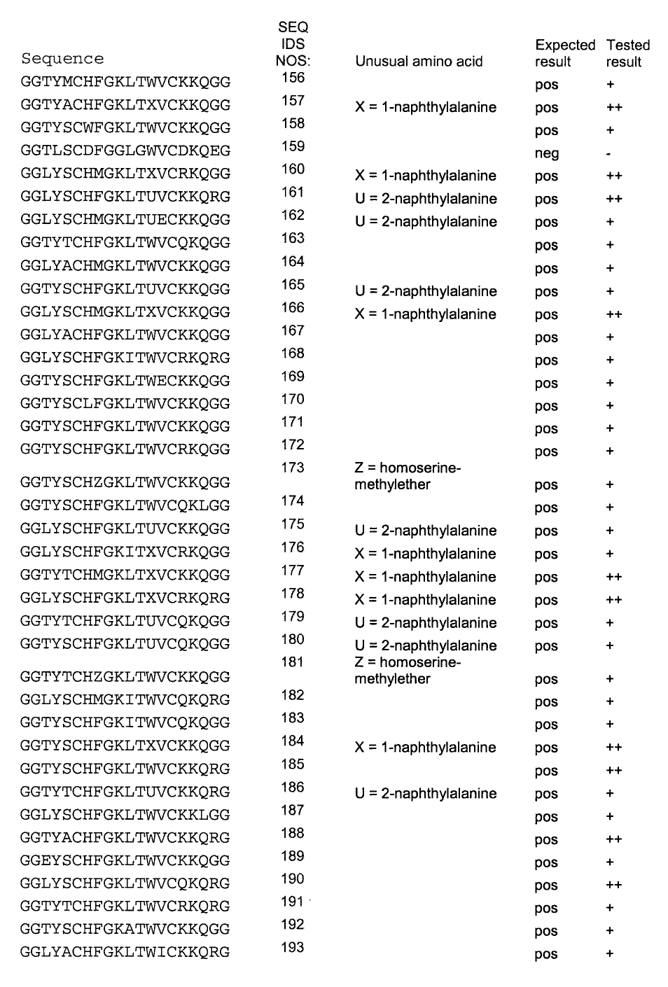

Using graphical evaluations as shown in FIGS. 17 and 18 based on calculations of the dose-response relationship using the program GraphPad the following EC50 values were determined on the basis of MTT-assay data:

The following table shows the EC₅₀ values of some exemplary peptides:

| | | |
|---|---|---|
| SEQ ID NO 2: | GGTYSCHFGKLTWVCKKQGG | 3284 nmol/1 |
| SEQ ID NO 4: | GGTYSCHFGKLTWVCKPQGG | 4657 nmol/1 |
| SEQ ID NO 5: | GGTYSCHFGRLTWVCKPQGG | 5158 nmol/1 |
| SEQ ID NO 6: | GGTYSCHFGRLTWVCKKQGG | 4969 nmol/1 |
| SEQ ID NO 7: | GGTYSCHF-(Als)-LTWVCKPQGG | 5264 nmol/1 |
| SEQ ID NO 8: | GGTYSCHF-(Als)-LTWVCKKQGG | 4996 nmol/1 |
| | GGTYSCHFGPLTWVCKKQGG | 2518 nmol/1 |
| | GGTYSCHFAKLTWVCKKQGG | 5045 nmol/1 |
| | GGTYSCHFGGLTWVCKPQGG | no activity |
| | | detectable |

### IV. Synthesis of Bivalent EPO Mimetic Peptides

Automated synthesis of linear SEQ ID NO 11 (AGEM11)

The synthesis is carried out by the use of a Liberty microwave system (CEM) using Rink-Amide-Resin (substitution rate 0.19 mmol/g) in a scale of 0.25 mmol. Removal of Fmoc-groups is achieved by double treatment with 10 ml piperidine/DMF (1:3) and irradiation with 50 W for 10×10 sec. Coupling of amino acids is achieved by double treatment with a of 4 fold excess of amino acid in DMF PyBOP/HOBT/DIPEA as coupling additives and irradiation with 50 W for 5×30 sec. Between all irradiation cycles the solution is cooled by bubbling nitrogen through the reaction mixture. After deprotection and coupling, the resin is washed 6 times with 10 ml DMF. After the double coupling cycle all unreacted amino groups are blocked by treatment with a 10 fold excess of N-(2-Chlorobenzyloxycarbonyloxy)succinimide (0.2M solution in DMF) and irradiation with 50 W for 3×30 sec. After deprotection of the last amino acid, the peptide is acetylated by incubation with 0.793 ml of capping-solution (4.73 ml acetic anhydride and 8.73 ml DIEA in 100 ml DMSO) for 5 minutes. Before cleavage the resin is then washed 6 times with 10 ml DMF and 6 times with 10 ml DCM. Cleavage of the crude peptides is achieved by treatment with 5 ml TFA/TIS/EDT/H₂O (94/1/2.5/2.5) for 120 minutes under an inert atmosphere. This solution is filtered into 40 ml cold ether, the precipitate dissolved in acetonitrile/water (1/1) and the peptide is purified by RP-HPLC (Kromasil 100 C18 10 μm, 250×4.6 mm).

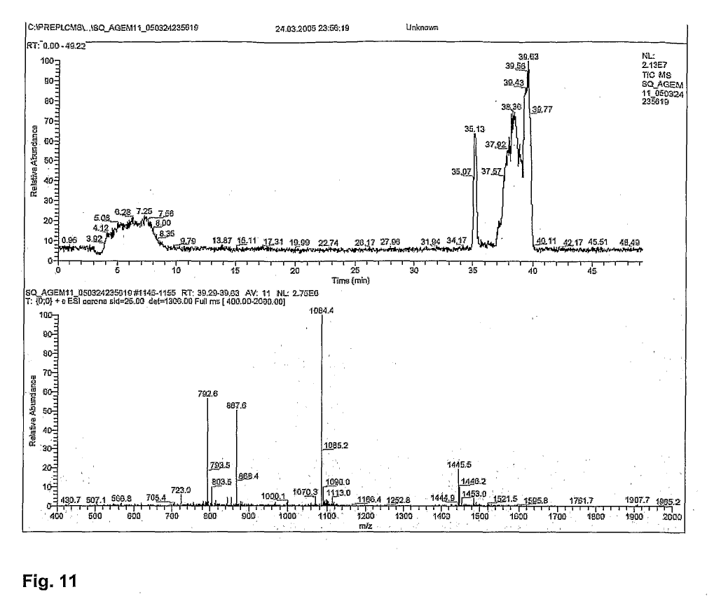

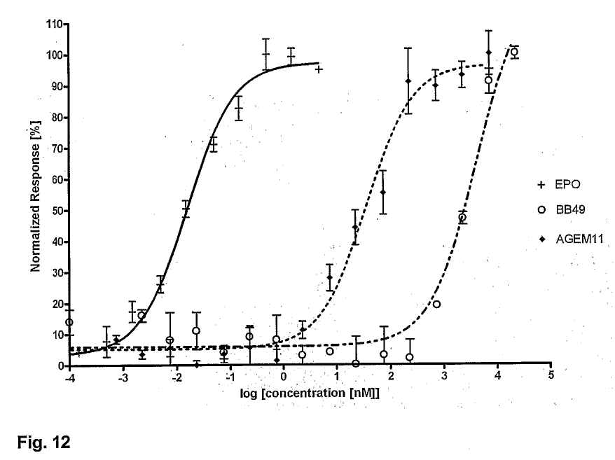

The purification scheme of linear AGEM11, Kromasil 100 C18 10 μm, 250×4.6 mm and the gradient used therefore is depicted in FIGS. 8 and 9 from 5% to 50% acetonitrile (0.1% TFA) in 50 minutes

Cyclization of linear AGEM11

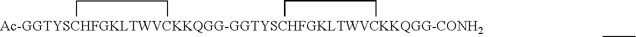

30 mg of the linear peptide are dissolved in 60 ml solution A. This solution und 60 ml DMSO are added dropwise to 60 ml solution A (total time for addition: 3 h). After 48 h the solvents are removed by evaporation and the remaining residue solved in 30 ml DMSO/water (1/1). 30 ml acetic acid and 17 mg iodine (solved in DMSO/water (1/1) are added and the solution is mixed for 90 minutes at room temperature. Afterwards 20 mg ascorbic acid are added and the solvents removed by evaporation. The crude mixture is solved in acetonitrile/water (2/1) and the peptide is purified by RP-HPLC (Kromasil 100 C18 10 μm, 250×4.6 mm).

Solution A: Acetonitrile/water (1/1) containing 0.1% TFA. The pH is adjusted to 8.0 by the addition of ammoniumbicarbonate.

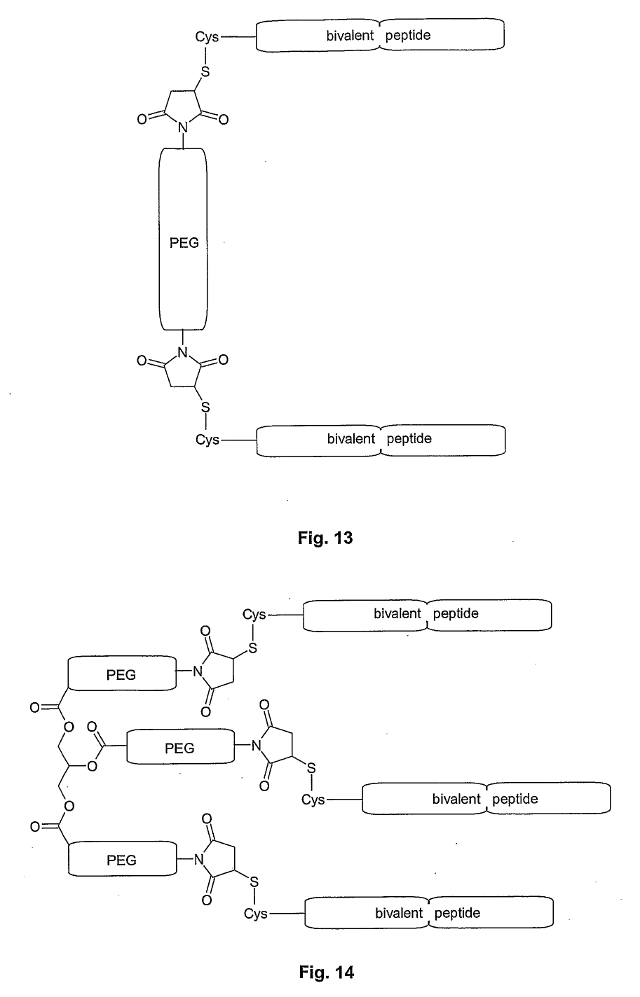

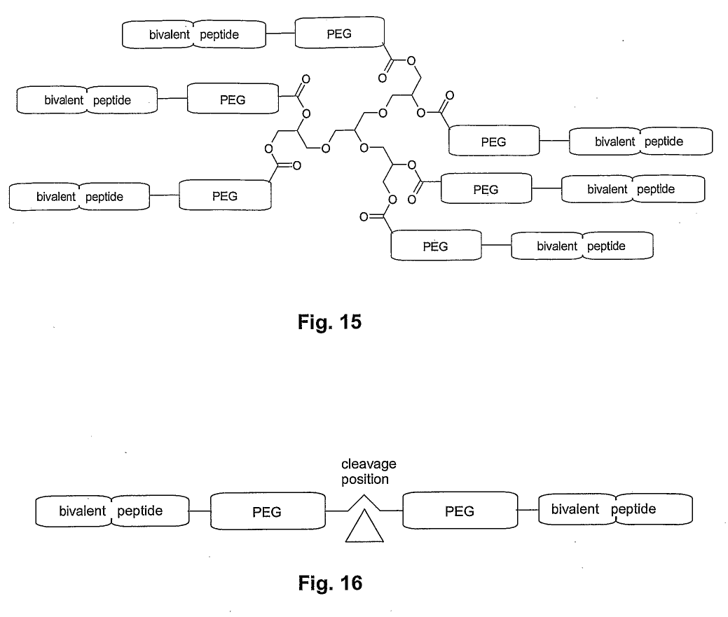

The purification parameters for cyclic AGEM11 are given in FIGS. 10 and 11 (scheme: Purification of cyclic AGEM11, Kromasil 100 C18 10 μm, 250×4.6 mm, gradient from 5% to 35% acetonitrile (0.1% TFA) in 50 minutes).

### V. In Vitro Proliferation Assay to Determine EPO Activity

TF1 cells in logarithmic growth phase (2.10⁵-1.10⁶ cells/ml grown in RPMI with 20% fetal calf serum (FCS) and 0.5 ng/ml IL-3) were counted, and the number of cells needed to perform an assay were centrifuged (5 min. 1500 rpm) and resuspended in RPMI with. 5% FCS without IL-3 at 300 000 cells/ml. Cells were precultured in this (starvation) medium without IL-3 for 48 hours. Before starting the assay the cells were counted again.

Shortly before starting the assay stock solutions of peptides and EPO were prepared. Peptides were weighed and dissolved in RPMI with 5% FCS up to a concentration of 1 mM, 467 μM or 200 μM. EPO stock solutions were 10 nM or 20 nM. Twohundredninetytwo μl of these stock solutions were pipetted into one well of a 96 well culture plate—one plate was taken for each substance to be tested. Twohundred μl of RPMI with 5% FCS were pipetted into seventeen other wells in each plate. Ninetytwo μl of stock solution were pipetted into a well containing 200 μl medium. The contents were mixed, and 92 μl from this well was transferred to the next, and so forth. This way a dilution series (18 dilutions) of each substance was prepared such that in each consecutive well the concentration was 1:√10 of the concentration in the well before that. From each well 3×50 μl was transferred to three empty wells. This way each concentration of substance was measured in quadruplicate. Note that the uppermost and lowermost line of wells of each plate was left void.

Pretreated (starved) cells were centrifuged (5 min. 1500 rpm) and resuspended in RPMI with 5% FCS at a concentration of 200 000 cells per ml. Fifty μl of cell suspension (containing 10 000 cells) was added to each well. Note that due to the addition of the cells the final concentrations of the substances in the wells were half that of the original dilution range. Plates were incubated for 72 h at 37° C. in 5% CO₂.

Before starting the evaluation, a dilution range of known amounts of TF-1 cells into wells was prepared: 0/2500/5000/10000/20000/50000 cells/well, were pipetted (in 100 μl RPMI+5% FCS) in quadruplicate.

To measure the number of live cells per well, ready-to-use MTT reagent (Promega, CellTiter 96 Aqueous One Solution Cell Proliferation Assay) was thawed in a 37° C. water bath. Per well, 20 μl of MTT reagent was added, and plates were incubated at 37° C. in 5% CO₂ for another 1-2 h. Twentyfive ∥l of a 10% SDS solution was added, and plates were measured in an ELISA reader (Genios, Tecan). Data were processed in spreadsheets (Excel) and plotted in Graphpad.

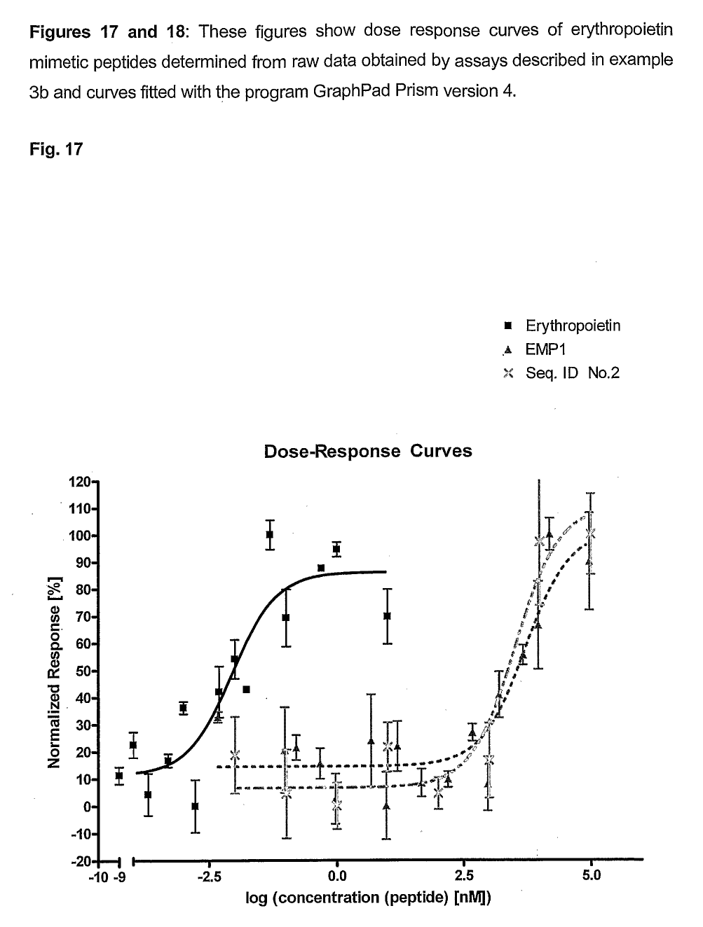

The data are summarized in FIG. 12.

| | ED50 (nM): |
|---|---|
| EPO | 0.0158 |
| BB49 (monomer, SEQ. ID NO 2) | 4113 |
| AGEM11 (bivalent) | 36.73 |

### VI. Extended Peptide Assays

In an extended assay, approximately 200 peptide sequences were tested for their EPO mimetic activity.

The peptides were synthesized as peptides amides on a LIPS-Vario synthesizer system. The synthesis was performed in special MTP-synthesis Plates, the scale was 2 μmol per peptide. The synthesis followed the standard Fmoc-protocol using HOBT as activator reagent. The coupling steps were performed as 4 times coupling. Each coupling step took 25 min and the excess of amino acid per step was 2.8. The cleavage and deprotection of the peptides was done with a cleavage solution containing 90% TFA, 5% TIPS, 2.5% H₂O and 2.5% DDT. The synthesis plate containing the finished peptide attached to the resin was stored on top of a 96 deep well plate. 50 μl of the cleavage solution was added to each well and the cleavage was performed for 10 min, this procedure was repeated three times. The cleaved peptide was eluted with 200 μl cleavage solution by gravity flow into the deep well plate. The deprotection of the side chain function was performed for another 2.5 h within the deep well plate. Afterwards the peptide was precipitated with ice cold ether/hexane and centrifuged. The peptides were solved in neutral aqueous solution and the cyclization was incubated over night at 4° C. The peptides were lyophilized.

FIG. 19 gives an overview over the synthesised and tested peptides monomers.

The peptides were tested for their EPO mimetic activity in an in vitro proliferation assay. The assay was performed as described under V. On each assay day, 40 microtiter plates were prepared for measuring in vitro activity of 38 test peptides, 1 reference example, and EPO in parallel. EPO stocks solutions were 20 nM.

The results are given in FIG. 19. As can be seen from the results, the tested peptides not fulfilling the consensus of the present invention did not depict EPO mimetic activity.

### VII. Synthesis of Peptide HES-Conjugates

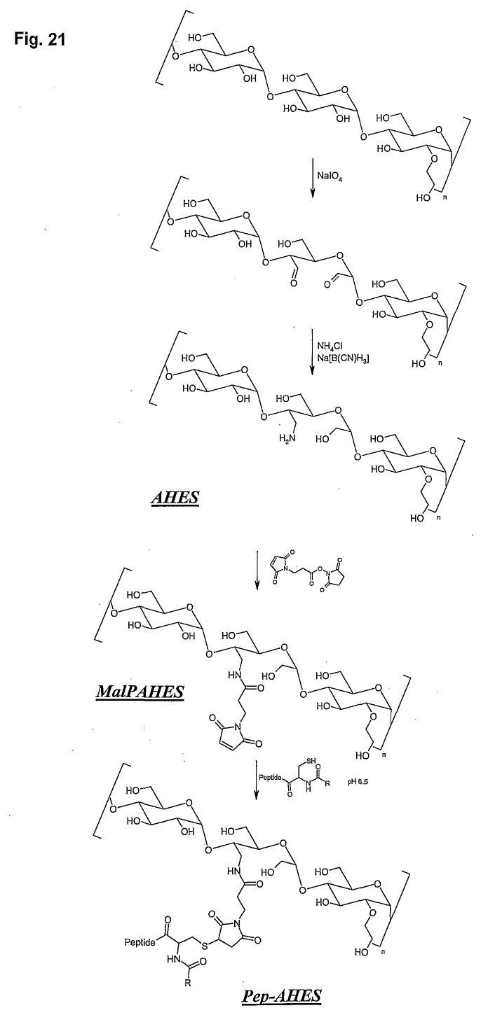

The principle reaction scheme is depicted in FIG. 21.

The aim of the described method, is the production of a derivative of a starch, according to this example HES, which selectively reacts with thiol groups under mild, aqueous reaction conditions. This selectivity is reached with maleimide groups.

HES is functionalised first with amino groups and converted afterwards to the respective maleimide derivative. The reaction batches were freed from low molecular reactants via ultra membranes. The product, the intermediate products as well as the educts are all poly-disperse.

### Synthesis of Amino-HES (AHES)

Hydroxyethylstarch (Voluven®) was attained via diafiltration and subsequent freeze-drying. The average molar weight was approximately 130 kDa with a substitution grade of 40%.

The synthesis was performed according to the synthesis described for amino dextran in the dissertation of Jacob Piehler, “Modifizierung von Oberflächen für die thermodynamische und kinetische Charakterisierung biomolekularer Erkennung mit optischen Transducern”, 1997, herein incorporated by reference. HES was activated by partial, selective oxidation of the diolic hydroxyl groups to aldehyde groups with sodium periodate as described in Floor et. al (1989). The aldehyde groups were converted via reductive amination with sodiumcyanoborhydride (NaCNBH₃) in the presence of ammonia to amino groups (Yalpani and Brooks, 1995).

Periodate opening: The used amount of periodate represents 20% of the number of glucose building blocks (applying a glucose building block mass of 180 g/mol, DS=0.4). The working-up was performed via ultra filtration and freeze-drying

Reductive animation with NH₄Cl/Na[BH₃CN] (in excess)

Working-up via precipitation of the product and dia-filtration.

### Analysis

Qualitative: Ninhydrin reaction (Kaiser-test)

Quantitative: with 2,4,6-trinitrobenzole sulphonic acid (TNBS) in comparison with an amino dextrane.

The achieved substitution grade was around 2.8%. This results in a molar mass of one building block carrying one amino group of approx. 6400 g/mol.

Synthesis of maleimidopropionyl-amino-hydroxyethylstarch (“MaIPA-HES”)

### Synthesis

3-maleimidopropione acid-N-hydroxysuccinimidester (MalPA-OSu) was used in excess (10-fold) (50 mM phosphate buffer, pH 7, 20% DMF, over night), working-up via ultra filtration and freeze-drying.

### Analysis

The reaction of the amino group was verified with ninhydrin and TNBS.

The number of introduced maleimide groups is demonstrated by reaction of gluthation (GSH) and the detection of excessive thiol groups with Ellmans reagent (DNTB) and via 700 MHz-¹H-NMR-spectroscopy

The achieved substitution grade was around 2% and corresponds to 8500 g/mol per maleimide building block (180 g/mol glucose building block mass, DS=0.4).

### Peptide-Hydroxyethylstarch-Conjugate (Pep-AHES)

### Synthesis

A cysteine containing peptide was used which had either a free (Pep-1A) or a biotinylated (Pep-1B) N-term. A 4:1 mixture of Pep-IA/B was converted over night in excess (approx. 6 equivalents with MaIPA-HES in phosphate-buffer, 50 mM, pH 6.5/DMF 80:20; working up occurred with ultra filtration and freeze-drying.

### Analysis

The UV-absorption was determined at 280 nm and the remaining, content of maleimide groups was determined with GSH/DNTB.

The peptide yield was almost quantitative. Nearly no free maleimide groups were detectable.

### VIII. Antibody-Cross Reactivity Assay

As described in the introduction of this application, patients sometimes develop antibodies against rhuEPO. This leads to the severe consequences described in the introduction.

In order to further explore the properties of the peptides according to the invention it was analysed whether the peptides in fact cross-react with anti-EPO antibodies.

Rabbit and human sera containing anti-EPO antibodies were used for testing.

These sera were pre-treated either with EPO or the following EPO mimetic peptides:

Ac-C-GGTYSCHFGKLT-1 nal-VCKKQRG-GGTYSCHFGKLT-1nal-VCKKQRG-Am (test peptide 1)
Ac-GGTYSCHFGKLT-1nal-VCKKQRG-Am (test peptide 2)
Ac=acetylated N-terminus
Am=amidated C-terminus
1nal=1-naphthylalanine

Different concentrations of erythropoietin and EPO mimetic peptides were used in the analysis. After pre-treatment of the sera with the test substances in order to adsorb the anti-EPO antibodies present in the sera, the sera were treated with radioactively labelled erythropoietin. The antibodies remaining in the sera after the pre-adsorption step are bound by the erythropoietin and again immunoprecipated. The protocol used for this test is described in Tacey et al., 2003, herein incorporated by reference.

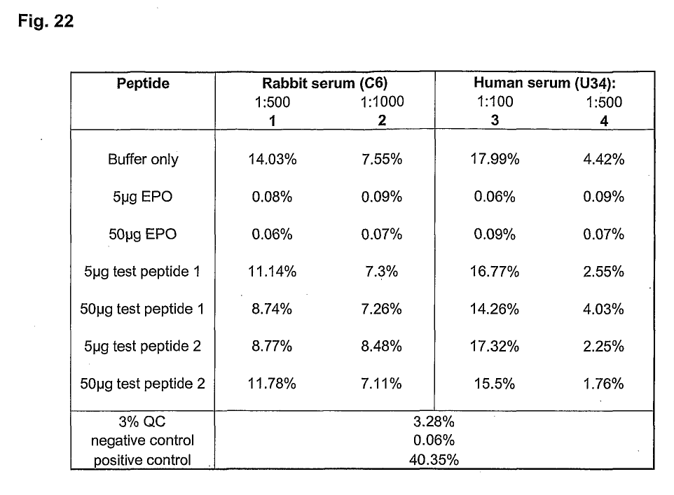

The results of the performed pre-adsorption with the anti-EPO antibody containing sera using either EPO or EPO mimetic peptides according to the invention are disclosed in FIG. 22.

When the sera were pre-treated with EPO mimetic peptides, the sera were afterwards tested positive when contacted with radioactively labelled erythropoietin. Thus anti-EPO antibodies were detected in the sera notwithstanding the pre-treatment. This means that the EPO mimetic peptides were not able to bind to the anti-EPO antibodies during pre-treatment. In the absence of a binding activity, the anti-EPO antibodies were not eliminated from the sera together with the EPO mimetic peptides and thus remained in the sera. The anti-EPO antibodies were not able to recognize and thus bind to the EPO mimetic peptides.

Recombinant human EPO (rhuEPO) was used as a control. When the sera were pre-treated with erythropoietin, pretty much no antibodies were detectable in the subsequent assay incorporating radioactively labelled erythropoietin since the antibodies were already bound and eliminated by the pre-treatment with erythropoietin.

The numerical values depicted in FIG. 22 represent the % cpm of the total counts used in the IP. A serum is assessed as positive when the % cpm value is >0.9. 100% cpm represents the amount of the overall used counts (the radioactive tracer), presently the radioactively labelled EPO.

The assay demonstrates that the EPO mimetic peptides according to the invention depict advantageously no cross-reactivity to anti-EPO antibodies. The EPO mimetic peptides described herein should thus depict a therapeutic effect even in patients who developed antibodies against rhuEPO. Furthermore, it is expected, that antibodies against EPO mimetic peptides should not bind erythropoietin. The EPO mimetic peptides according to this invention are thus preferably also characterised in that they show no significant cross-reactivity with anti-EPO antibodies.

### REFERENCES

Wrighton N C, Balasubramanian P, Barbone F P, Kashyap A K, Farrell F X, Jolliffe L, Barrett R W, Dower W J (1997) Increased potency of an erythropoietin peptide mimetic through covalend dimerization. Nature Biotechnology 15:1261-1265
Wrighton N C, Farrell F X, Chang R, Kashyap A K, Barbone F P, Mulcahy L S, Johnson D L, Barrett R W, Jolliffe L K, Dower W J (1996) Small Peptides as Potent Mimetics of the Protein Hormone Erythropoietin. Science 273:458-463
Johnson, D. L., F. X. Farrell, et al. (1997). “Amino-terminal dimerization of an erythropoietin mimetic peptide results in increased erythropotietic activity.” Chemistry and Biology 4: 939-950.
Haag R, Sunder A, Stumbe J F, J. Am. Chem. Soc. (2000), 122, 2954.
Roberts, M. J., M. D. Bentley, et al. (2002). “Chemistry for peptide and protein PEGylation.” Advanced Drug Delivery Review 54(4): 459-476.
Richard Tacey, Anthony Greway, Janice Smiell, David Power, Arno Kromming a, Mohamed Daha, Nicole Casadevall and Marian Kelley: The detection of anti-erythropoietin antibodies in human serum and plasma—Part I. Validation of the protocol for a radioimmunoprecipitation assay; J Immunol Methods. 2003 December; 283(1-2):317-29.
Zalipsky S, Qazen, S, Walker II J A, Mullah N, Quinn Y P, (1999) “New detachable poly(ethylene glycol) conjugates: Cysteine-cleavable lipopolymers regenerating natural phospholipid, diacyl phosphatidylethanolamine, Bioconjug. Chem. 10: 703-707.
Zhao, X. et al (1997), “Novel Degradable Poly(ethylene glycol) esters for drug delivery.” In “Poly(ethylene glycol) chemistry and biological applications; Harris J M, Zalipsky, S. Eds.; ACS Symposium Series 680; American Chemical Society: Washington D.C., 1997; 458-472.

1. A peptide comprising at least 10 amino acids, wherein the peptide has EPO receptor agonist activity, wherein the peptide does not have proline in the position referred to as position 10 of EPO mimetic peptides, but a positively charged amino acid.

2. The peptide according to claim 1, comprising an amino acid motif, wherein the amino acid motif is a beta-turn motif.

3. The peptide according to claim 1, wherein the amino acids at positions 9 and 10 are substituted by a single amino acid, which is 5-aminolevulinic acid (5-Als):

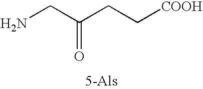

4. The peptide according to claim 1, wherein the peptide has a positively charged amino acid in position 17.

5. A peptide comprising the following sequence of amino acids:

| | |
|---|---|
| X₆X₇X₈X₉X₁₀X₁₁X₁₂X₁₃X₁₄X₁₅; | (SEQ ID NO: 1) |

wherein the peptide has EPO receptor agonist activity,
wherein X represents an amino acid and each amino acid is chosen from natural and unnatural amino acids and
X₆ is C, A, E, α-amino-γ-bromobutyric acid or homocysteine (hoc);
X₇ is R, H, L, W or Y or S;
X₈ is M, F, I, homoserinemethylether or norisoleucine;
X₉ is G or a conservative exchange of G;
X₁₀ is a non conservative exchange of proline;
or X₉ and X₁₀ are substituted by a single amino acid;
X₁₁ is selected from any amino acid;
X₁₂ is T or A;
X₁₃ is W, 1-nal, 2-nal, A or F;
X₁₄ is D, E, I, L or V; and
X₁₅ is C, A, K, α-amino-γ-bromobutyric acid or homocysteine (hoc),
provided that either X₆ or X₁₅ is C or hoc.

6. The peptide according to claim 5,
(SEQ ID NO: 241)
X₆X₇X₈X₉X₁₀X₁₁X₁₂X₁₃X₁₄X₁₅; wherein
X₆ is C;
X₇ is R, H, L or W;
X₈ is M, F or I;
X₉ is G or a conservative exchange of G;
X₁₀ is a non conservative exchange of proline;
X₁₁ is independently selected from any amino acid;
X₁₂ is T;
X₁₃ is W;
X₁₄ is D, E, I, L or V; and
X₁₅ is C;
or X₆X₇X₈X₉X₁₀X₁₁X₁₂X₁₃X₁₄X₁₅; (SEQ ID NO: 242) wherein X₉ and X₁₀ are substituted by a single amino acid;
or wherein
X₆ is C;
X₇ is R, H, L or W;
X₈ is M, F, I, or hsm (homoserine methylether);
X₉ is G or a conservative exchange of G;
X₁₀ is a non conservative exchange of proline;
X₁₁ is independently selected from any amino acid;
X₁₂ is T;
X₁₃ is W;
X₁₄ is D, E, I, L or V, 1-nal (1-naphthylalanine) or 2-nal (2-naphthylalanine); and
X₁₅ is C.

7. The peptide according to claim 5, wherein X₁₀ is an amino acid with a positively charged side chain or X₉ and X₁₀ are substituted by a single amino acid chosen from 5-aminolevulinic acid (Als) and aminovaleric acid.

8. The peptide according to claim 5 comprising further amino acids as shown in the sequence;
(SEQ ID No: 3)
X₄X₅X₆X₇X₈X₉X₁₀X₁₁X₁₂X₁₃X₁₄X_{15,}
or
(SEQ ID No: 4)
X₃X₄X₅X₆X₇X₈X₉X₁₀X₁₁X₁₂X₁₃X₁₄X₁₅X₁₆X₁₇X₁₈
wherein X₃ is chosen from any amino acid;
X₄ is Y;
X₅ is chosen from any amino acid;
X₁₆ is chosen from any amino acid;
X₁₇ is chosen from any amino acid; and
X₁₈ is chosen from any amino acid.

9. The peptide according to claim 8,
wherein
X₃ is chosen from D, E, L, N, S, T and V;
X₄ is Y;
X₅ is chosen from A, H, K, L, M, S, T and l;
X₁₆ is chosen from G, K, L, Q, R, S and T;
X₁₇ is chosen from A, G, P, R, K, Y and homoarginine; and
X₁₈ is any amino acid.

10. The peptide according to claim 9, wherein at least one of the following conditions is met: X₆ is C, E, A or hoc; X₇ is R, S, H or Y; X₈ is F or M; X₉ is F or A; X₁₀ is K or Har; X₁₁ is V, L, I, M, E, A, T or norisoleucine; X₁₂ is T; X₁₃ is W; X₁₄ is D or V; X₁₅ is C or Hoc; X₁₇ is P, Y, A, K, or Har.

11. The peptide according to claim 5 comprising an amino acid sequence chosen from the group consisting of:
SEQ ID NO [2] 205: GGTYSCHFGKLTWVCKKQGG;
SEQ ID NO [4] 206: GGTYSCHFGKLTWVCKPQGG;
SEQ ID NO [7] 209: GGTYSCHF-(Als)-LTVVVCKPQGG;
and
SEQ ID NO [8] 210: GGTYSCHF-(Als)-VPNVCKKQGG,
wherein
Als is the amino acid residue of 5-aminolevulinic acid (Als), which is represented by the formula:

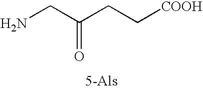

12. A peptide comprising an amino acid sequence chosen from the group consisting of:
(SEQ ID NO: 207)
GGTYSCHFGRLTWVCKPQGG;
(SEQ ID NO: 208)
GGTYSCHFGRLTWVCKKQGG;
(SEQ ID NO: 211)
GGTYSCHFGKLT-1naL-VCKKQRG;
(SEQ ID NO: 212)
GGTYSCHFGKLTWVCKKQGG-GGTYSCHFGKLTWVCKKQGG;
(SEQ ID NO: 219)
GGTYSCHFGKLT-1nal-VCKKQRG-GGTYSCHFGKLT-1nal-
VCKKQRG;
(SEQ ID NO: 220)
CGGTYSCHFGKLTWVCKKQGG-GGTYSCHFGKLTWVCKKQGG;
(SEQ ID NO: 221)
CGGTYSCHFGKLT-1nal-VCKKQRG-GGTYSCHFGKLT-1nal-
VCKKQRG;
(SEQ ID NO: 222)
GGTYSCSFGKLTWVCK-Har-QGG;
(SEQ ID NO: 223)
GGTYSCHFG-Har-LTWVCK-Har-QGG;
(SEQ ID NO: 6)
GGTYSCHMGKLTXVCKKQGG;
(SEQ ID NO: 8)
GGTYTCHFGKLTXVCKKLGG;
(SEQ ID NO: 19)
GGLYSCHFGKITXVCKKQGG;
(SEQ ID NO: 24)
GGLYSCHMGKLTWVCRKQGG;
(SEQ ID NO: 33)
GGLYSCHFGKLTXVCQKQGG;
(SEQ ID NO: 34)
GGTYSCHFGKLTWVCQKQRG;
(SEQ ID NO: 40)
GGTYSCHFGKLTXVCKKQRG;
(SEQ ID NO: 42)
GGLYACHFGKLTWDCQKQGG;
(SEQ ID NO: 47)
GGTYTCHFGKLTUVCKKQGG;
(SEQ ID NO: 51)
GGTYSCHFGKLTUVCKKLGG;
(SEQ ID NO: 66)
GGTYSCHFGKITXVCKKQGG;
(SEQ ID NO: 74)
GGLYSCHFGKLTUVCKKLGG;
(SEQ ID NO: 76)
GGLYACHFGKLTUVCKKQGG;
(SEQ ID NO: 88)
GGLYSCHMGKLTWLCKKLGG;
(SEQ ID NO: 101)
GGTYSCRFGKLTWVCKKQGG;
(SEQ ID NO: 106)
GGTYTCHFGKITUVCKKQGG;
(SEQ ID NO: 108)
GGLYSCHFGKLTXVCKKQGG;
(SEQ ID NO: 111)
GGLYACHFGKLTULCKKQGG;
(SEQ ID NO: 120)
GGLYSCHFGKLTWVCKKQRG;
(SEQ ID NO: 125)
GGTYTCHFGKITXVCKKQGG;
(SEQ ID NO: 126)
GGTYTCHMGKLTWVCKKQRG;
(SEQ ID NO: 243)
GGLYSCHFGKLTXVCKKQRG;
(SEQ ID NO: 135)
GGTYTCHFGKLTXVCKKQGG;
(SEQ ID NO: 144)
GGLYSCHFGKITUVCKKQGG;
(SEQ ID NO: 145)
GGLYSCHFGKLTXVCRKQGG;
(SEQ ID NO: 153)
GGTYACHFGKLTXVCKKLGG;
(SEQ ID NO: 154)
GGLYACHFGKLTXVCRKQGG;
(SEQ ID NO: 157)
GGTYACHFGKLTXVCKKQGG;
(SEQ ID NO: 160)
GGLYSCHMGKLTXVCRKQGG;
(SEQ ID NO: 161)
GGLYSCHFGKLTUVCKKQRG;
(SEQ ID NO: 166)
GGLYSCHMGKLTXVCKKQGG;
(SEQ ID NO: 177)
GGTYTCHMGKLTXVCKKQGG;
(SEQ ID NO: 178)
GGLYSCHFGKLTXVCRKQRG;
(SEQ ID NO: 184)
GGTYSCHFGKLTXVCKKQGG;
(SEQ ID NO: 185)
GGTYSCHFGKLTWVCKKQRG;
(SEQ ID NO: 188)
GGTYACHFGKLTWVCKKQRG;
(SEQ ID NO: 190)
GGLYSCHFGKLTWVCQKQRG;
and
(SEQ ID NO: 197)
GGTYTCHFGKLTXVCKKQRG,
wherein X is 1-naphthylalanine and U is 2-naphthylalanine.

13. (canceled)

14. A peptide according to claim 5, wherein said peptide does not cross-react with anti-EPO antibodies.

15. A peptide according to claim 5, wherein said peptide is modified, wherein the peptide comprises at least one modification chosen from: N-terminal acetylation (Ac); C-terminal acetylation (Ac); N-terminal amidation (Am); C-terminal amidation (Am); intramolecular cyclisation; phosphorylation; N-methylglycine (meG); N-acetylglycine (AcG); and attaching a polymeric moiety to the peptide.

16-18. (canceled)

19. A synthetic peptide comprising a continuous peptide chain comprising at least two domains, wherein at least one domain comprises a peptide according to claim 5.

20-21. (canceled)

22. A peptide according to claim 27, wherein said linking moiety comprises 3 to 5 amino acid residues chosen from glycine, alanine, and alanine derivates.

23. (canceled)

24. A peptide comprising a continuous peptide chain comprising at least two domains, wherein at least one domain comprises a peptide, wherein said peptide comprises a peptide sequence chosen from:
(SEQ ID NO: 212)
GGTYSCHFGKLTWVCKKQGG-GGTYSCHFGKLTWVCKKQGG;
(SEQ ID NO: 219)
GGTYSCHFGKLT-1nal-VCKKQRG-GGTYSCHFGKLT-1nal-
VCKKQRG;
and peptides according to claim 12, wherein said peptide optionally has a cysteine at the N-terminus, and wherein said peptide optionally comprises an intramolecular disulfide bridge.

25. A peptide dimer or multimer comprising
a first peptide;
a second peptide; and
optionally additional peptides; and
optionally a linking moiety connects said first and second peptide,
wherein each additional peptide, when present, is independently and optionally connected to the previous peptide via a linking moiety,
wherein at least one of said peptides comprises a peptide according to claim 5.

26. A peptide dimer or multimer according to claim 25, wherein the C-terminus of said first peptide is covalently bound to the N-terminus of said second peptide or the C-terminus of said peptide is covalently bound to the C-terminus of said second peptide or the N-terminus of said first peptide is covalently bound to the N-terminus of said second peptide.

27. A peptide dimer or multimer according to claim 25, wherein said linking moiety comprises a sequence of natural and/or non-natural amino acids.

28. A peptide dimer or multimer according to claim 25, wherein said linking moiety comprises a diketopiperazine unit.

29-30. (canceled)

31. A peptide according to claim 5, further comprising a water soluble polymer covalently bound to said peptide, the water soluble polymer is chosen from the group consisting of polyethylene glycol, dextrans and starches.

32. The peptide according to claim 31, wherein said water soluble moiety is PEG, having a molecular weight of at least 10 kD.

33. A compound comprising:
(i) a peptide according to claim 19; and
(ii) at least one polymeric carrier unit;
wherein said peptides are attached to said polymeric carrier unit.

34. The compound according to claim 33, wherein said carrier unit comprises at least one natural or synthetic branched, dendritic or linear polymer chosen from the group consisting of polyglycerines, polysialine acid, dextrans, starches, polyethylene glycol, and other biologically inert water soluble polymers.

35-37. (canceled)

38. The compound according to claim 33, wherein said carrier unit comprises at least two polymeric subunits, wherein said polymeric subunits are connected to each other via at least one biodegradable covalent linker structure.

39. The compound according to claim 33, comprising a first biodegradable carrier unit, wherein said peptides and said second polymeric carrier units are attached to said first biodegradable polymeric carrier unit.

40-48. (canceled)

49. A pharmaceutical composition comprising a compound according to claim 5 and optionally a pharmaceutical acceptable carrier.

50. A method for the treatment of a disorder comprising administering to a patient in need an effective amount of a composition according to claim 49, wherein the disorder is chosen from: a deficiency of erythropoietin; a low or defective red blood cell population; a disorder treatable by the administration of erythropoietin; any type of anemia; and stroke.

51-53. (canceled)

54. A nucleic acid encoding for a peptide according to claim 5.

55. (canceled)
